(19) United States
(12) Patent Application Publication
(10) Pub. No.: US 2024/0197815 A1
(43) Pub. Date: Jun. 20, 2024

(54) METHODS AND COMPOSITIONS FOR IMPROVING VISUAL FUNCTION IN OCULAR DISEASES AND DISORDERS

(71) Applicant: ONL Therapeutics, Inc., Ann Arbor, MI (US)

(72) Inventors: Andrew J. KOCAB, Farmington Hills, MI (US); Constance I. CHANG, Ann Arbor, MI (US); David N. ZACKS, Ann Arbor, MI (US); David A. ESPOSITO, Portage, MI (US); Jana VAN DE GOOR, Jerome, MI (US); David KLEINMAN, Rochester, NY (US); Lindsay M. GODSEY, Grosse Pointe Woods, MI (US); Sushanta MALLICK, Richardson, TX (US)

(21) Appl. No.: 18/410,820

(22) Filed: Jan. 11, 2024

Related U.S. Application Data
(63) Continuation of application No. PCT/US22/36830, filed on Jul. 12, 2022.
(60) Provisional application No. 63/221,329, filed on Jul. 13, 2021.
(60) Provisional application No. 63/336,896, filed on Apr. 29, 2022.

(51) Int. Cl. A61K 38/10; A61P 27/02

(52) U.S. Cl. CPC A61K 38/10; A61P 27/02

(57) ABSTRACT

Provided herein are methods and compositions for improving visual function in an eye of an individual having an ocular disorder. The methods described herein generally comprise methods of improving visual function (e.g., BCVA) in an eye of an individual having an ocular disease or disorder, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual.

### CROSS REFERENCE

This application is a continuation of National Application No. PCT/US2022/036830, filed Jul. 12, 2022, which claims the benefit of U.S. Provisional Patent Application No. 63/221,329 filed June Jul. 13, 2021, and U.S. Provisional Patent Application No. 63/336,896 filed Apr. 29, 2022, each of which is incorporated by reference in entirety for all purposes.

### SEQUENCE LISTING

The instant application contains a Sequence Listing which has been submitted electronically in XML format and is hereby incorporated by reference in its entirety. Said XML copy, created on Oct. 28, 2022, is named 58109711601_s1 and is 44,229 bytes in size.

### BACKGROUND

Vision is generally dependent on maintaining the integrity of the structures of the eye, and changes in retinal homeostasis can provide the basis for vision loss and/or retinal diseases or disorders. Therapies for the treatment of ocular diseases and disorders generally target stopping or reducing the deleterious changes associated ocular dysfunction resulting from diseases or disorders of the eye.

### SUMMARY

Targeting a cessation or a reduction of deleterious changes to visual function associated with ocular disease can be useful in treatment programs, however, restoration and improvement of visual function represents a more preferred and, in general instances, more difficult to achieve patient outcome. Described and provided herein are compositions and methods useful for improving visual function in an eye of an individual having an ocular disease and/or disorder.

Provided and described herein are methods of improving visual function (e.g., BCVA) in an eye of an individual having an ocular disease or disorder, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual. In some embodiments, the ocular disease or disorder comprises a loss in visual function. In some embodiments, the ocular disease or disorder comprises photoreceptor cell death or a symptom thereof (e.g., a loss in visual function). In some embodiments, the ocular disease or disorder comprises retinal pigment epithelial cell death or a symptom thereof (e.g., a loss in visual function). In some embodiments, the ocular disease or disorder comprises degeneration of the macula.

In some embodiments, the visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, the method comprises improving visual function without performing surgery.

In some embodiments, the method further comprises performing surgery (e.g., standard of care surgery) on the eye of the individual. In some embodiments, the surgery is performed after administering the peptide or the variant sequence thereof, or the pharmaceutically-acceptable salt of the peptide. In some embodiments, the surgery comprises retinal reattachment surgery. In some embodiments, the surgery comprises a vitrectomy.

In some embodiments, improving visual function comprises improving visual function as compared to a baseline visual function prior to the administering peptide or the variant sequence thereof, or the pharmaceutically-acceptable salt of the peptide.

Provided and described herein are methods of treating macular degeneration having geographic atrophy in an eye of an individual, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual. Provided and described herein are methods of treating a loss in visual function (e.g., BCVA) in an eye of an individual having macular degeneration, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual.

In some embodiments, treating comprises improving visual function. In some embodiments, improving visual function is compared to a baseline visual function prior to administering the peptide or the variant sequence thereof, or the pharmaceutically-acceptable salt of the peptide. In some embodiments, the macular degeneration comprises age-related macular degeneration. In some embodiments, the macular degeneration comprises geographic atrophy.

In some embodiments, visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and/or light/dark adaptation.

Provided and described herein are methods of treating retinal detachment in an eye of an individual, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Provided and described herein are methods of treating a loss in visual function in an eye of an individual having a retinal detachment, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual.

In some embodiments, treating comprises inhibiting a deterioration and/or a loss of visual acuity in the eye. In some embodiments, treating comprises improving visual function. In some embodiments, improving visual function is compared to a baseline visual function prior to administering the peptide or the variant sequence thereof, or the pharmaceutically-acceptable salt of the peptide. In some embodiments, treating comprises inhibiting photoreceptor cell death.

In some embodiments, the retinal detachment comprises a fully detached retina or a partially detached retina. In some embodiments, the retinal detachment comprises a fully detached retina. In some embodiments, the retinal detachment comprises a partially detached retina. In some embodiments, the surgery comprises retinal reattachment surgery. In some embodiments, the surgery comprises a vitrectomy.

In some embodiments, the eye of the individual has a retina that is detached, and wherein the retina has been detached for about (y) or more days (e.g., prior to administering the composition comprising the peptide), wherein (y) is at least one. In some embodiments, (y) is 1 to 21. In some embodiments, (y) is 3 to 21. In some embodiments, (y) is 7 to 21.

In some embodiments, surgery is performed about (z) day(s) after administering the composition comprising the peptide, wherein (z) is at least one. In some embodiments, (z) is 1. In some embodiments, (z) is 1 to 3. In some embodiments, (z) is 1 to 7.

In some embodiments, the method comprises administering the peptide or the variant sequence thereof, or the pharmaceutically-acceptable salt of the peptide to the vitreous of the eye. In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, the modification comprises a modified amino acid. In some embodiments, the peptide comprises an amidated C-terminus. In some embodiments, the peptide has the structure of Formula I or a pharmaceutically-acceptable salt thereof. In some embodiments, the peptide has the structure of Formula III or a pharmaceutically-acceptable salt thereof.

In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, the peptide or the variant sequence thereof, or the pharmaceutically-acceptable salt of the peptide is formulated in a composition. In some embodiments, the composition further comprises one or more excipients. In some embodiments, the composition further comprises a surfactant. In some embodiments, the surfactant is a non-ionic surfactant. In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant forms about 0.01% to about 20% weight/weight of the composition. In some embodiments, the surfactant forms about 0.05% to about 10% weight/weight of the composition. In some embodiments, the composition further comprises a tonicity adjusting agent, a buffering agent, or a combination thereof. In some embodiments, the composition is buffered at a pH of 2.5 to 7.5.

In some embodiments, 5 micrograms (ug) to 10,000 ug of the peptide or the variant sequence thereof, or the pharmaceutically-acceptable salt of the peptide is administered. In some embodiments, the peptide or the variant sequence thereof, or the pharmaceutically-acceptable salt of the peptide at least 10 micrograms (ug), at least 25 ug, at least 50 ug, at least 100 ug, at least 150 ug, at least 200 ug, or at least 250 ug of the peptide is administered. In some embodiments, the peptide is present at a concentration 0.1 milligrams per milliliter (mg/mL) to 10.0 mg/mL.

Further provided herein are methods of improving visual function when combined with surgery (e.g., retinal re-attachment and/or a vitrectomy). For example, provided herein are methods and compositions for use in treating detachment of the neural retina and/or inhibiting the deterioration and/or loss of visual acuity after detachment of the neural retina. In some embodiments, the methods provided herein utilize administering a Fas inhibitor (e.g., Fas inhibitor peptide) (e.g., to the eye of an individual, such as vitreous thereof) and performing surgery (e.g., retinal reattachment surgery). In specific embodiments, the Fas inhibitor is administered to the eye of individual (e.g., the vitreous thereof), such as wherein the eye has (or is suspected of having) a (e.g., partial or full) detached retina (e.g., full or partially detached). In some instances, a method provided herein (e.g., comprising administering a Fas inhibitor to the eye of an individual) inhibits inflammation of an eye of an individual, such as an eye having a detached retina (e.g., full or partially detached). By way of further example, in certain instances, administration of the Fas inhibitor to the eye (e.g., by inhibiting inflammation in an eye having a detached neural retina and performing surgery (e.g., retinal reattachment surgery) inhibits the deterioration of visual acuity resulting from and/or associated with detachment of the neural retina (e.g., compared to surgery alone). In certain instances, the methods and compositions provided herein are useful for improving patient outcomes (e.g., inhibiting deterioration of visual acuity) in patients having a detached neural retina (e.g., when compared to surgery alone).

Provided in certain embodiments herein are methods (e.g., of treating an ocular disorder), the methods comprising administering (e.g., a composition comprising) a Fas inhibitor to an eye of an individual. Provided in some embodiments herein are methods (e.g., of treating an ocular disorder), the methods comprising: administering (e.g., a composition comprising) a Fas inhibitor to an eye of an individual and performing surgery (e.g., standard of care surgery) on the eye of the individual. Provided in specific embodiments herein are methods (e.g., of treating an ocular disorder) comprising: administering (e.g., a composition comprising) a peptide comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

In some embodiments, provided herein is a method of treating a retinal detachment in the eye of an individual, the method comprising administering a Fas inhibitor (e.g., a peptide comprising an amino acid sequence HHIYLGAVNYIY or a variant sequence thereof) to the eye of the individual and (e.g., subsequently) performing surgery (e.g., standard of care surgery) on the eye of the individual (e.g., the surgery at least partially re-attaching the retina). In certain embodiments, retinal detachment is rhegmatogenous retinal detachment (RRD). In certain embodiments, retinal detachment is macula-off rhegmatogenous retinal detachment (RRD). In certain embodiments, retinal detachment is macula-on rhegmatogenous retinal detachment (RRD). In certain embodiments, retinal detachment is tractional retinal detachment. In certain embodiments, retinal detachment is exudative detachment. In certain embodiments, retinal detachment is a result of retinal injury.

In some embodiments, the method is a method of treating a loss and/or deterioration in visual acuity. In some embodiments, the method is a method of inhibiting, reducing, and/or preventing a loss and/or deterioration in in visual acuity. In some embodiments, the method is method of treating a loss and/or deterioration in visual function. In some embodiments, the method is a method of inhibiting, reducing, and/or preventing a loss and/or deterioration in visual function.

In some embodiments, the method is a method of treating photoreceptor cell loss in an eye. In some embodiments, the method is a method of inhibiting, reducing, and/or preventing photoreceptor cell loss in an eye. In some embodiments, the method is a method of treating inflammation (e.g., Fas-mediated inflammation) in an eye. In some embodiments, the method is a method of inhibiting, reducing, and/or preventing inflammation (e.g., Fas-mediated inflammation) in an eye. In some embodiments, the method is a method of treating an ocular disease, disorder, or condition. In some embodiments, the method is a method of inhibiting progression of an ocular disease, disorder, or condition, or symptoms associated therewith.

In some embodiments, the surgery comprises a vitrectomy, a scleral buckle, a pneumatic retinopexy, a laser surgery, or a combination thereof. In some embodiments, the surgery comprises a vitrectomy. In certain embodiments, the vitrectomy comprises the use of silicone oil (e.g., the vitreous is replaced by silicone oil). In certain embodiments, the vitrectomy comprises the use of gas (e.g., the vitreous is replaced by a gas). In some embodiments, the surgery comprises a scleral buckle. In some embodiments, the surgery comprises a pneumatic retinopexy. In some embodiments, the surgery comprises a laser surgery. In some embodiments, the surgery comprises or a combination of one or more of the following: a vitrectomy, a scleral buckle, a pneumatic retinopexy, and a laser surgery.

In some embodiments, the individual has a detached neural retina. In certain embodiments, the neural retina is fully detached. In certain embodiments, the neural retina is partially detached. In certain embodiments, the neural retina is fully detached. In some embodiments, the extent of detachment is between 0-1, greater about 1, greater than about 2, greater than about 3, greater than about 4, greater than about 5, greater than about 6, greater than about 7, greater than about 8, greater than about 9, greater than about 10, greater than about 11, or greater than about 12 (on a scale based on clock hours, ranging from 0-12). In some embodiments, the height of detachment (as measured from the central macula) is between 0 and 1 millimeter(s), greater than about 1 millimeter(s), greater than about 2 millimeter(s), greater than about 3 millimeter(s), greater than about 4 millimeter(s), or greater than about 5 millimeter(s).

In some embodiments, surgery is performed about (z) day(s) after administering the composition comprising the peptide, wherein (z) is at least one. In certain embodiments, (z) is 1, 2, 3, 4, 5, 6, 7, 8, 9, 10, or a number greater than 10. In some embodiments, surgery is performed about (h) hour(s) after administering the composition comprising the peptide, wherein (h) is at least one. In certain embodiments, (h) is 1, 2, 3, 4, 5, 6, 12, 18, 24, 30, or 36.

In some embodiments, the individual has baseline (e.g., prior to treatment) best corrected visual acuity (BCVA) of detecting hand motion. In some embodiments, the individual has baseline (e.g., prior to treatment) best corrected visual acuity of counting fingers. In some embodiments, the individual has baseline (e.g., prior to treatment) BCVA of 20/100 (e.g., from a Snellen chart, ETDRS, Tumbling C, HOTV for young children, etc.). In some embodiments, the individual has baseline (e.g., prior to treatment) BCVA of 3 or greater. In some embodiments, the individual has baseline (e.g., prior to treatment) BCVA of logMAR 2 or greater. In some embodiments, the individual has baseline (e.g., prior to treatment) BCVA of logMAR 1 or greater. In some embodiments, the individual has baseline (e.g., prior to treatment) BCVA of logMAR 0.5 or greater.

In some embodiments, the individual has baseline (e.g., prior to treatment) BCVA of 20/200 or greater. In some embodiments, the individual has baseline (e.g., prior to treatment) BCVA of 20/100 or greater. In some embodiments, the individual has baseline (e.g., prior to treatment) BCVA of 20/70 or greater. In some embodiments, the individual has baseline (e.g., prior to treatment) BCVA of 20/50 or greater.

In some embodiments, the individual has baseline (e.g., prior to treatment) visual angle in minutes of 32 or greater. In some embodiments, the individual has baseline (e.g., prior to treatment) BCVA in visual angle in minutes of 24 or greater. In some embodiments, the individual has baseline (e.g., prior to treatment) BCVA in visual angle in minutes of 16 or greater. In some embodiments, the individual has baseline (e.g., prior to treatment) BCVA in visual angle in minutes of 8 or greater. In some embodiments, the individual has baseline (e.g., prior to treatment) BCVA in visual angle in minutes of 4 or greater. In some embodiments, the individual has baseline (e.g., prior to treatment) BCVA in visual angle in minutes of 2 or greater.

In some embodiments, the subject has central vision loss. In some embodiments, the eye of the individual has a retina that is detached, and wherein the retina has been detached for about (y) or more days (e.g., prior to administering the composition comprising the Fas inhibitor or performing surgery), wherein (y) is at least one. In some embodiments, the eye of the individual has central vision loss, and wherein the central vision loss has been present for about (y) or more days (e.g., prior to administering the composition comprising the Fas inhibitor or performing surgery), wherein (y) is at least one. In certain embodiments, the eye of the individual has a retinal injury or a symptom thereof, and wherein the retinal injury or the symptom thereof has been present for about (y) or more days (e.g., prior to administering the composition comprising the Fas inhibitor or performing surgery), wherein (y) is at least one. In certain embodiments, (y) is 1 (e.g., about 1 or more days), 2, 3, 4, 5, 6, 7, 8, 14, 21, 28, or a number greater than 28.

In some embodiments, the subject has symptoms associated with a detached neural retina (e.g., a loss or reduction in visual function). In some embodiments, the eye of the individual has symptoms associated with a detached neural retina (e.g., a loss or reduction in visual function), and wherein the symptoms associated with a detached neural retina (e.g., a loss or reduction in visual function) have been present for about (s) or more days (e.g., prior to administering the composition comprising the Fas inhibitor or performing surgery), wherein (s) is at least one. In certain embodiments, (s) is 1 (e.g., about 1 or more days), 2, 3, 4, 5, 6, 7, 8, 14, 21, 28, or a number greater than 28.

In some embodiments, the individual is 40 years of age or older. In some embodiments, the individual is about 50 years of age or older. In some embodiments, the individual is about 55 years of age or older. In some embodiments, the individual is about 60 years of age or older. In some embodiments, the individual is about 65 years of age or older. In some embodiments, the individual is 70 years of age or older.

In some embodiments, the eye of the individual comprises inflammation (e.g., an increased amount of one or more biomarkers).

In some embodiments, the eye of the individual has a cataract. In some embodiments, the eye of the individual potentially has a cataract. In some embodiments, the eye of the individual is at risk for developing a cataract (e.g., showing one or more symptoms of a cataract, being advanced in age, experiencing trauma to the eye, etc.). In some embodiments, the eye of the individual potentially has a pseudophakic lens. In some embodiments, the individual has a preexisting disease or disorder that includes non-ocular pathologies (e.g., diabetes).

In some embodiments, the composition is administered via intravitreal injection, intracameral injection, injection into the suprachoroidal space, injection in the subtenons space, subconjunctival injection, retrobulbar injection, peribulbar injection, microneedle injection, subretinal injection, or subretinal infusion.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, comprises a modified amino acid or a non-natural amino acid. In some embodiments, the peptide comprises an amidated C-terminus, wherein the peptide has the structure of Formula I, or a pharmaceutically-acceptable salt thereof.

In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, the composition further comprises on or more excipients. In some embodiments, the composition further comprises a surfactant. In some embodiments, the surfactant is a non-ionic surfactant.

In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant is a polysorbate. In some embodiments, the surfactant is a polyethoxylated alcohol. In some embodiments, the surfactant is a polyoxyethylene-polyoxypropylene block copolymer. In some embodiments, the surfactant is an oxyethylated tertiary octylphenol formaldehyde polymer.

In some embodiments, the surfactant comprises Polysorbate 20, Poloxamer 407, Tyloxapol, or cremophor. In some embodiments, the surfactant comprises Polysorbate 20. In some embodiments, the surfactant comprises Poloxamer 407. In some embodiments, the surfactant comprises Tyloxapol. In some embodiments, the surfactant comprises cremophor. In some embodiments, the surfactant forms about 0.01% to about 20% weight/weight of the composition. In some embodiments, the surfactant forms about 0.05% to about 10% weight/weight of the composition. In some embodiments, the non-ionic surfactant is about 0.05% w/w of the composition to about 2% w/w of the composition. In some embodiments, the non-ionic surfactant is at least about 0.05% w/w of the composition.

In some embodiments, the composition further comprises a tonicity adjusting agent, a buffering agent, or a combination thereof (e.g., as described herein, including any of the described ranges and/or values within the described ranges). In some embodiments, the composition is buffered at a pH of 2.5 to 7.5.

In some embodiments, the composition comprises 5 micrograms (ug) to 10,000 ug of the peptide. In some embodiments, the composition comprises about 5 micrograms (ug) of the peptide to about 300 ug of the peptide. In some embodiments, the peptide is present at a concentration 0.1 milligrams per milliliter (mg/mL) to 10.0 mg/mL.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. shows data demonstrating improved visual acuity outcomes in GA patients receiving a Fas inhibitor.

FIG. 2A-2B provides exemplary conversions of measures of visual acuity. FIG. 2A shows a first exemplary conversion table of measures of visual acuity. FIG. 2B shows a second exemplary conversion table of measures of visual acuity.

### DETAILED DESCRIPTION

Described and provided herein are compositions and methods useful for improving visual function in an eye of an individual having an ocular disease and/or disorder. In certain instances, improvement of visual function is achieved by treating (e.g., inhibiting, reducing, and/or preventing) Fas-related inflammation in the eye, thereby preventing, reducing, and/or inhibiting retinal degeneration. Retinal degeneration is a complex, multifactorial condition where cell death (e.g., cell apoptosis), glial cell activation, and inflammation in or surrounding retinal tissue are linked to the death of cells within the retina (e.g., retinal pigment epithelial (RPE) cells and/or photoreceptors (PRs)). Fas-mediated inflammation can directly or indirectly lead to retinal degeneration and can be associated with the symptoms resulting from retinal degeneration. The Fas-mediated inflammation signaling pathway is generally initiated by an interaction between the membrane-bound Fas ligand (FasL—a type II transmembrane protein of the TNF family) and Fas receptor, thereby leading to the activation of pro-inflammatory signaling (e.g., cytokine signaling, interleukin signaling, caspase action, etc.) and/or cell death signaling (e.g., apoptotic signaling, necrotic signaling, etc.) pathways.

### Fas Inhibitors

Provided herein are Fas inhibitors useful for modulating (e.g., inhibiting, preventing, and/or reducing, etc.) Fas-mediated signaling. In certain instances, the Fas inhibitors useful in treating, inhibiting, preventing, and/or reducing Fas-mediated inflammation. In certain instances, inhibiting, preventing, and/or reducing Fas-mediated inflammation allows for the treatment and/or prevention of cell loss within the retina (e.g., photoreceptors and/or retinal pigment epithelium). Accordingly, in certain instances, the Fas inhibitors are also useful in treating and/or preventing retinal cell loss and/or the symptoms associated with retinal cell loss. In certain instances, inhibiting, preventing, and/or reducing Fas-mediated inflammation further treats, inhibits, reduces, and/or prevents retinal cell loss (e.g., retinal epithelial (RPE) cells and/or photoreceptor (PR)). cells). In some embodiments, the Fas inhibitors described herein are useful for treating, inhibiting, reducing, and/or preventing retinal cell loss and/or the symptoms associated with retinal cell loss. In such instances, because the Fas inhibitors described herein are useful in the manner described above, the Fas inhibitors are useful in method of treating ocular diseases and disorders.

In some embodiments, the Fas inhibitors described herein encompass Met-derived peptides and/or fragments thereof. In some embodiments, the Met protein, also called c-Met or hepatocyte growth factor receptor (HGF receptor), is encoded by the Met gene (NCBI Gene ID 4233, Location: NC_000007.14 (116672196.116798386), UniProtKB—P0858). The Met protein is comprised of two major subunits: the a and p subunits, and Met and fragments of Met, including the extracellular domain of Met and its a subunit, have been shown to bind to Fas and prevent cells from undergoing apoptosis. In some embodiments, the Fas inhibitor comprises a Fas-inhibiting peptide (e.g., Met-derived peptide and/or fragment thereof). In some embodiments, the Fas inhibitors described herein comprises a Met-derived compound comprising the amino acid acids HHIYLGAVNYIY (His-His-Ile-Tyr-Leu-Gly-Ala-Val-Asn-Tyr-Ile-Tyr) (e.g., SEQ ID NOs: 1-8). In some embodiments, the peptide comprises the amino acid sequence HHIYLGAVNYIY or a variant sequence thereof.

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the Fas inhibitor peptides (e.g., a peptide comprising the amino acid sequence HHIYLGAVNYIY) comprises one or more naturally occurring amino acids. In some embodiments, the Fas inhibitor peptides (e.g., a peptide comprising the amino acid sequence HHIYLGAVNYIY) consists of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the Fas inhibitor comprises a variant sequence of the peptide (e.g., a peptide comprising the amino acid sequence HHIYLGAVNYIY). In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, the variant sequence comprises one amino acid substitution. In some embodiments, the variant sequence comprises two amino acid substitutions. In some embodiments, the variant sequence comprises three amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In some embodiments, the Fas inhibitor peptides (e.g., a peptide comprising the amino acid sequence HHIYLGAVNYIY) comprises one or more non-natural amino acids. In some embodiments, the Fas inhibitor peptides (e.g., a peptide comprising the amino acid sequence HHIYLGAVNYIY) consists of non-natural amino acids. As used herein, non-natural amino acids and/or unnatural amino acids include and/or refer to amino acid structures that cannot be generated biosynthetically in any organism using unmodified or modified genes from any organism. In some embodiments, non-natural amino acids and/or unnatural amino acids further include and/or refer to an amino acid residue that are not present in the naturally occurring (wild-type) Met protein sequence. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring (wild-type) Met protein sequence. A non-natural amino acid residue also can be incorporated such that a desired functionality is imparted to the apelin molecule, for example, the ability to link a functional moiety (e.g., PEG).

In some embodiments, a variant sequence comprises one or more amino acid deletions. In some embodiments, the variant sequence comprises one amino acid deletion. In some embodiments, the variant sequence comprises two amino acid deletions. In some embodiments, the variant sequence comprises three amino acid deletions. In some embodiments, the variant sequence comprises four amino acid deletions. In some embodiments, the variant sequence comprises one or more additional amino acids. In some embodiments, the additional amino acids are additional amino acids from the Met sequence. In some embodiments, the variant sequence comprises a substitution and a deletion. In some embodiments, the variant sequence comprises a substitution and one or more additional amino acids. In some embodiments, the substitution comprises a natural amino acid or a non-natural amino acid. In some embodiments, the variant sequence is a retro inverso amino acid sequence. In some embodiments, a variant sequence comprises one or more additional amino acid residues (e.g., one, two, or three additions) to the N or C terminus. In some embodiments, a variant sequence comprises one or more deletions (e.g., one, two, or three deletions) to amino acid residues at the N or C terminus.

Functionality of variant sequences of the peptide (e.g., a variant sequence of the amino acid sequence HHIYLGAVNYIY) can be determined by an in vitro assay. For example, in some embodiments, the variant sequence competes for binding to a Fas receptor (FasR) with Fas ligand (FasL). In some embodiments, the variant sequence inhibits, reduces, or prevents caspase 8 activation in cells treated with FasL (e.g., as measured by commercially available luminescent tetrapeptide cleavage assay kit (Promega, Madison, WI)). In some embodiments, the variant sequence inhibits, reduces, or prevents cell death of cells treated with FasL. By way of further example, in some embodiments, the variant sequence competes for binding to a Fas receptor (FasR) with a Fas-activating antibody (e.g., Fas-agonistic Jo2 monoclonal antibody (BD Biosciences, San Jose, CA)). In some embodiments, the variant sequence inhibits, reduces, or prevents caspase 8 activation in cells treated with a Fas-activating antibody (e.g., as measured by commercially available luminescent tetrapeptide cleavage assay kit (Promega, Madison, WI)). In some embodiments, the variant sequence inhibits, reduces, or prevents cell death of cells treated with a Fas-activating antibody. Accordingly, in some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with Fas ligand (FasL). In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents caspase 8 activation in cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents cell death of cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents caspase 8 activation in cells treated with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents cell death of cells treated with a Fas-activating antibody.

The peptide or a variant sequence thereof can further comprise one or more modifications. In some embodiments, the peptide (e.g., a comprising the amino acid sequence HHIYLGAVNYIY or a variant sequence thereof) comprises a modification. In some embodiments, the peptide is a modified peptide. As used herein, a modification or a modified peptide includes and/or refers to a modification of one or more amino acids in the peptide. In some embodiments, modifications species of stereoisomers. All stereoisomers of the above compounds are contemplated, either in admixture or in pure or substantially pure form. The compounds can have asymmetric centers at any of the atoms. Consequently, the peptide compounds or components thereof can exist in enantiomeric or diastereomeric forms or in mixtures thereof. The present invention contemplates the use of any racemates (i.e., mixtures containing equal amounts of each enantiomer), enantiomerically enriched mixtures (i.e., mixtures enriched for one enantiomer), pure enantiomers or diastereomers, or any mixtures thereof. The chiral centers can be designated as R or S or R, S or d, D, 1, L or d, 1, D, or L. Compounds comprising amino acid residues include residues of D-amino acids, L-amino acids, or racemic derivatives of amino acids. Compounds comprising sugar residues include residues of D-sugars, L-sugars, or racemic derivatives of sugars. Non-limiting examples of modifications are phosphorylation, glycosylation, ubiquitination, nitrosylation, methylation, acetylation, amidation, or lipidation. Modification can be introduced at the C-terminus of the peptide, the N-terminus of the peptide, or at any place in-between. Thus, a modification or a modified peptide includes and/or refers to modifications of the free amino- and/or carboxyl-terminal (N-terminus and C-terminus, respectively). In some embodiments, N-terminal modifications include but are not limited to acetylation, formylation, pyroglutamylation, carbamide addition, lipidation, sulfonamidation, and alkylamination. In some embodiments, C-terminal modifications include but are not limited to amidation, esterification, and incorporation of an aldehyde group. In some embodiments, the modification comprises amidation. In some embodiments, the amidation is at the c-terminus. In some embodiments, the modification comprises a retro inverso peptide (e.g., YIYNVAGLYIHH). In some embodiments, the modification altering the chirality of one or more amino acid residues of the peptide (e.g., L amino acid to D amino acid).

Accordingly, in an embodiment, provided herein are peptides comprising the sequence (a)-HHIYLGAVNYIY-(b) or (a)-YIYNVAGLYIHH-(b), or a variant sequence thereof, wherein:
(a) is —H, —OH, —NH₂, G¹(CH₂)ₙ—, R¹CONH—, or R²O—;
(b) is —H, —CH₂OH, —CH₂OR², —CHO, —CO₂R², —CONH₂, —CONHR², —CON(R³)₂, —CONH(CH₂)_{y}NR(³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂, NH-Glu-His-OH, NH-Glu-His-NH₂, -Ala-His-NH₂, -Gly-His-NH₂, —NH-Glu-His-OH, —NH-Glu-His-NH₂, -Ala-His-NH₂, -Gly-His-NH₂, —NH-[D]Glu-[D]-His-OH, —NH-[D]Glu-[D]-His-NH₂, -[D]Ala-[D]-His-NH₂, -Gly[D]-His-NH₂, or —CONH(CH₂)˜-G²; G¹, at each occurrence, is independently —H, —C(═O)NH₂, —C(═O)NHR², —C(═O)N(R³)₂, C(═O)OR², or —C(═O)R¹; G² at each occurrence is a heterocyclic ring of 4-7 members comprising at least one tertiary amine functionality NR² within the ring, or a carbocyclic ring of 3-7 members substituted with —N(R³)₂;
R¹, at each occurrence, is independently H, C₁₋₆alkyl, —(CH₂)ₓ(OCH₂CH₂)ₘOR⁵, C₁₋₆-alkoxy or L;
R², at each occurrence, is independently C₁₋₆alkyl, C₂₋₆alkyl substituted with OR⁵ or NR⁵², —(CH₂)ₓ(OCH₂CH₂)ₘOR⁵ or L;
L, at each occurrence, is a multivalent polyethylene glycol derivative with 2-4 termini, each of which may be independently capped with H, R⁵;
R³, at each occurrence, is independently C₁₋₆alkyl, C₂₋₆alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆alkyl, C₁₋₆acyl, or —OPO₃(R⁵)₂;
R⁵, at each occurrence, is independently H or C₁₋₆alkyl;
R⁶, at each occurrence, is H, C₁₋₆alkyl, C₂₋₆hydroxyalkyl, C₁₋₆alkoxy-, C₁₋₆alkyl, or C₁₋₆acyl;
m=1-100;
n=0-3;
x=0-6; and
y=2-4,
wherein at most one of R¹ and R² is L.

In certain instances, provided herein are peptides comprising the structure of Formula I or Formula II, or a pharmaceutically-acceptable salt thereof.

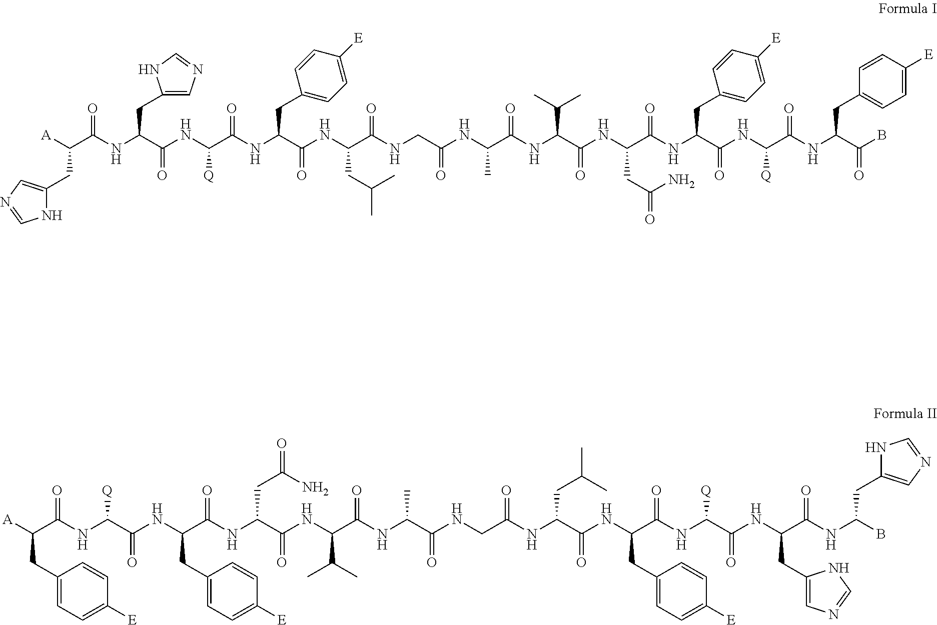

wherein:
   A is H—, —OH, —NH₂, G¹(CH₂)ₙ—, R¹CONH—, or R²O—;
   B is —H, CH₂OH, CH₂OR², —CHO, —CO₂R², —CONH₂, —CONHR², —CON(R³)₂, —CONH(CH₂)_{y}N(R³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂ NH-Glu-His-OH, NH-Glu-His-NH₂, -Ala-His-NH₂, -Gly-His-NH₂, NH-Glu-His-OH, NH-Glu-His-NH₂, -Ala-His-NH₂, -Gly-His-NH₂, NH-[D]Glu-[D]-His-OH, NH-[D]Glu-[D]-His-NH², -[D]Ala-[D]-His-NH₂, -Gly-[D]-His-NH₂, or CONH(CH₂)˜-G²;
   E, at each occurrence, is independently —H, —OH, —OR⁴, SH, —SR⁴, or halogen; G¹, at each occurrence, is independently —H, —C(═O)NH₂, —C(═O)NHR², —C(═O)N(R³)₂, C(═O)OR², or —C(═O)R¹;
   G² at each occurrence is a heteroalicyclic ring of 4-7 members comprising at least one tertiary amine functionality NR² within the ring, or a carbocyclic ring of 3-7 members substituted with N(R³)₂;
   Q, at each occurrence, is independently, 1-propyl, 2-propyl, 2-methyl-prop-2-yl, C₃₋₆-cycloalkyl, C₄₋₆-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran-4-yltetrahydrothiopyran-2-yl, tetrahydrothiopyran-3-yl, tetrahydrothiopyran-4-yl or 1-CH(OR⁵)CH₃;
   R¹, at each occurrence, is independently H, C₁₋₆alkyl, —(CH₂)ₓ(OCH₂CH₂)ₘOR⁵, C₁₋₆alkoxy or L;
   R², at each occurrence, is independently C₁₋₆alkyl, C₂₋₆alkyl substituted with OR⁵ or N(R⁵)₂, —(CH₂)ₓ(OCH₂CH₂)ₘOR⁵ or L; L, at each occurrence, is a multivalent polyethylene glycol derivative with 2-4 termini, each of which may be independently capped with H, R⁵ or another molecule of the peptide of Formula I or II;
   R³, at each occurrence, is independently C₁₋₆-alkyl, C₂₋₆-alkyl substituted with OR⁵ or N(R⁵)₂, —(CH₂)ₓ(OCH₂CH₂)ₘOR⁵;
   or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
   R⁴, at each occurrence, is independently C₁₋₆alkyl, C₁₋₆acyl, or —OPO₃(R⁵)₂;
   R⁵, at each occurrence, is independently H or C₁₋₆alkyl;
   R⁶, at each occurrence, is H, C₁₋₆alkyl, C₂₋₆hydroxyalkyl, C₁₋₆alkoxy-, C₁₋₆alkyl, or C₁₋₆acyl;
   m=1-100;
   n=0-3;
   x=0-6; and
   y=2-4,
   wherein at most one of R¹ and R² is L.

In some embodiments, provided is a peptide having the structure of Formula III or a pharmaceutically-acceptable salt thereof:

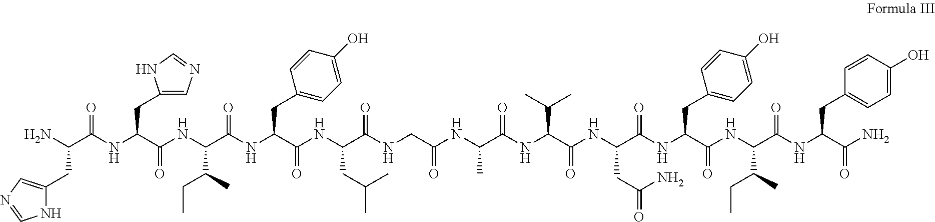 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

Formula III:
(SEQ ID NO: 3)
His-His-Ile-Tyr-Leu-Gly-Ala-Val-Asn-Tyr-Ile-
Tyr-amide.

In some embodiments, provided is a peptide having the structure of Formula IV or a pharmaceutically-acceptable salt thereof:

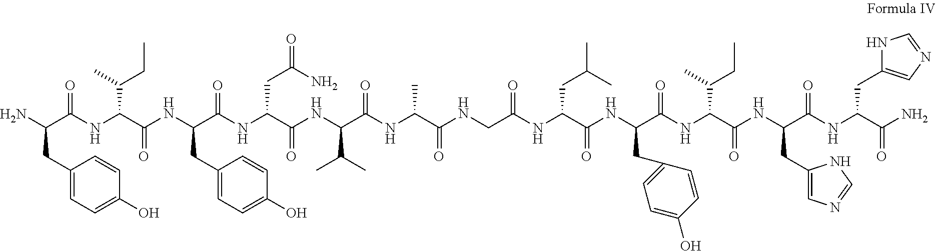 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

Formula IV:
(SEQ ID NO: 4)
All [D]Tyr-Ile-Tyr-Asn-Val-Ala-Gly-Leu-Tyr-
Ile-His-His-amide.

In some embodiments, provided is a peptide having the structure of Formula V or a pharmaceutically-acceptable salt thereof:

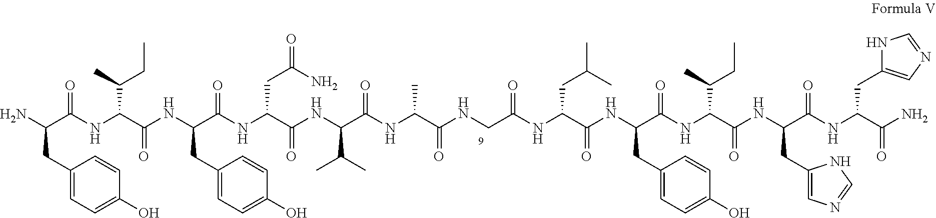 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

Formula V:
(SEQ ID NO: 5)
All [D]Tyr-allo-Ile-Tyr-Asn-Val-Ala-Gly-Leu-
Tyr-allo-Ile-His-His-amide.

In some embodiments, provided is a peptide having the structure of Formula VI or a pharmaceutically-acceptable salt thereof:

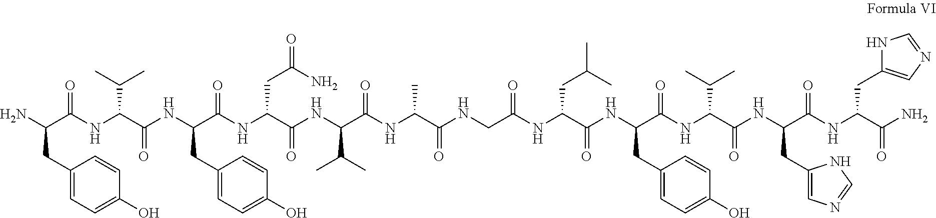 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

Formula VI:
(SEQ ID NO: 6)
All [D]Tyr-Val-Tyr-Asn-Val-Ala-Gly-Leu-Tyr-
Val-His-His-amide.

In some embodiments, provided is a peptide having the structure of Formula VII or a pharmaceutically-acceptable salt thereof:

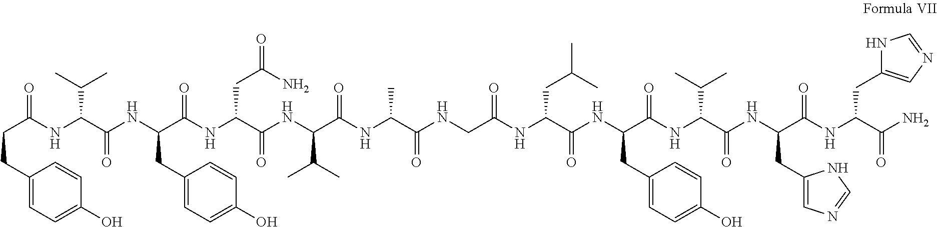

Formula 7:
(SEQ ID NO: 7)
All [D](DesaminoTyr)-Val-Tyr-Asn-Val-Ala-Gly-
Leu-Tyr-Val-His-His-amide.

In some embodiments, provided is a peptide having the structure of Formula VIII or a pharmaceutically-acceptable salt thereof:

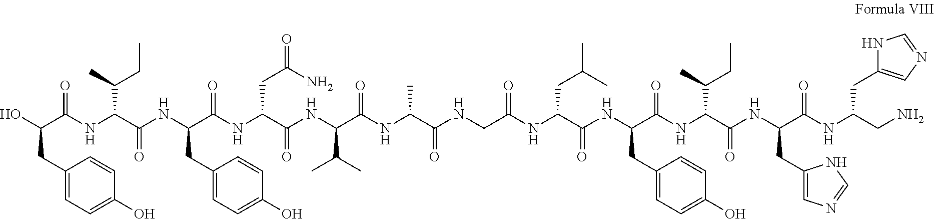 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@@H](CN)Cc1cnc[nH]1)[C@@H](C)CC)C(C)C]

Formula 8:
(SEQ ID NO: 8)
All [D](Hydroxy-desaminoTyr)-allo-Ile-Tyr-Asn-
Val-Ala-Gly-Leu-Tyr-allo-Ile-His-Histamine.

In some embodiments, provided is a peptide having the structure of Formula IX or a pharmaceutically-acceptable salt thereof:

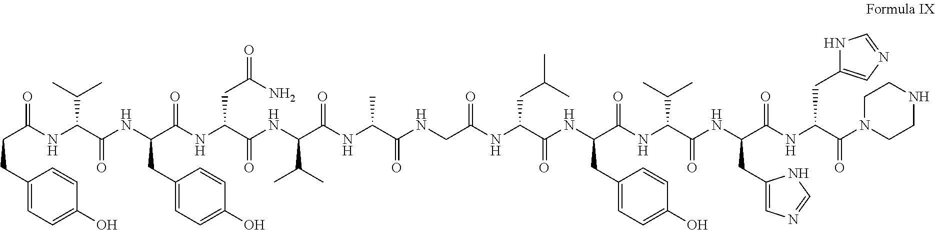

Formula IX:
(SEQ ID NO: 9)
All [D](DesaminoTyr)-Val-Tyr-Asn-Val-Ala-Gly-
Leu-Tyr-Val-His-His-piperazine amide.

### Salts of Fas Inhibiting Peptides

Further provided herein are salts of the peptide for inhibiting Fas-mediated inflammation in the eye and for use in the methods described here. As used herein, salt is generally synonymous with pharmaceutically-acceptable salts, and/or includes or refers to pharmaceutically-acceptable salts. Examples of pharmaceutically-acceptable salts are salts with organic or inorganic acids such as (but not limited to) include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared using methods well known in the art. In some embodiments, the salt is a hydrochloride salt.

In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, and polyamine resins.

Salts and pharmaceutically acceptable salts are described in in J. Pharmaceutical Sciences, 66: 1-19 (1977), the contents of which are incorporated by reference herein.

In some embodiments, the salt is an acetate salt. In some embodiments, the acetate salt is a poly-acetate salt. In some embodiments, the poly-acetate salt is a tri-acetate salt.

### Pharmaceutical Compositions

In an embodiment, further provided are pharmaceutical compositions (also referred to as compositions) comprising the Fas inhibiting peptides. In some embodiments, the pharmaceutical compositions described herein comprise the peptide (e.g., a peptide comprising the amino acid sequence HHIYLGAVNYIY) or a pharmaceutically-acceptable salt thereof. In some embodiments, the pharmaceutical compositions described herein comprise the peptide comprising the amino acid sequence HHIYLGAVNYIY or variant sequence thereof, or a pharmaceutically-acceptable salt of the peptide. In some embodiments, the pharmaceutical compositions described herein comprise the peptide having the structure of any one of Formulas I-IX or a pharmaceutically-acceptable salt thereof. In some embodiments, the pharmaceutical compositions described herein comprise the peptide having the structure of Formula I or a pharmaceutically-acceptable salt thereof. In some embodiments, the pharmaceutical compositions described herein comprise the peptide having the structure of Formula III or a pharmaceutically-acceptable salt thereof.

The pharmaceutical composition can comprise one or more excipients. As used herein, an excipient includes and/or refers to any pharmaceutically acceptable additive, carrier, diluent, adjuvant, or other ingredient, other than the active pharmaceutical ingredient (API), which is typically included for formulation and/or administration to a patient. A pharmaceutical composition can comprise a single pharmaceutical formulation (e.g., extended release, immediate release, delayed release, nanoparticulate, etc.) or multiple formulations (e.g., immediate release and delayed release, nanoparticulate and nonnanoparticulate, etc.). An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

In some embodiments, the compositions comprise excipients that are suitable for ocular application. Suitable excipients and include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1, and the like. Suitable chelating agents include sodium edetate and the like. Suitable buffering agents include phosphates, borates, citrates, acetates, tromethamine, and the like. Suitable surfactants include ionic and nonionic surfactants. In some embodiments, the one or more excipients comprises nonionic surfactants, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

In some embodiments, the composition comprises a non-ionic surfactant. In some embodiments, the composition comprises a polysorbate, a polyethoxylated castor oil derivative, a polyethoxylated fatty acid, a polyethoxylated alcohol, a polyoxyethylene-polyoxypropylene block copolymer (Poloxamer), or an oxyethylated tertiary octylphenol formaldehyde polymer (Tyloxapol). In some embodiments, the composition comprises a polysorbate. In some embodiments, the composition comprises a polyethoxylated castor oil derivative. In some embodiments, the composition comprises a polyethoxylated fatty acid. In some embodiments, the composition comprises a polyethoxylated alcohol. In some embodiments, the composition comprises a polyoxyethylene-polyoxypropylene block copolymer (Poloxamer). In some embodiments, the composition comprises an oxyethylated tertiary octylphenol formaldehyde polymer (Tyloxapol). In some embodiments, the surfactant makes up 0.05%-20% weight per weight (w/w) of the composition. In some embodiments, the non-ionic surfactant is about 0.05% w/w of the composition to about 20% w/w of the composition. In some embodiments, the non-ionic surfactant is at least about 0.05% w/w of the composition. In some embodiments, the non-ionic surfactant is at most about 20% w/w of the composition. In some embodiments, the non-ionic surfactant is about 0.05% w/w of the composition to about 0.1% w/w of the composition, about 0.05% w/w of the composition to about 0.5% w/w of the composition, about 0.05% w/w of the composition to about 1% w/w of the composition, about 0.05% w/w of the composition to about 2% w/w of the composition, about 0.05% w/w of the composition to about 5% w/w of the composition, about 0.05% w/w of the composition to about 10% w/w of the composition, about 0.05% w/w of the composition to about 20% w/w of the composition, about 0.1% w/w of the composition to about 0.5% w/w of the composition, about 0.1% w/w of the composition to about 1% w/w of the composition, about 0.1% w/w of the composition to about 2% w/w of the composition, about 0.1% w/w of the composition to about 5% w/w of the composition, about 0.1% w/w of the composition to about 10% w/w of the composition, about 0.1% w/w of the composition to about 20% w/w of the composition, about 0.5% w/w of the composition to about 1% w/w of the composition, about 0.5% w/w of the composition to about 2% w/w of the composition, about 0.5% w/w of the composition to about 5% w/w of the composition, about 0.5% w/w of the composition to about 10% w/w of the composition, about 0.5% w/w of the composition to about 20% w/w of the composition, about 1% w/w of the composition to about 2% w/w of the composition, about 1% w/w of the composition to about 5% w/w of the composition, about 1% w/w of the composition to about 10% w/w of the composition, about 1% w/w of the composition to about 20% w/w of the composition, about 2% w/w of the composition to about 5% w/w of the composition, about 2% w/w of the composition to about 10% w/w of the composition, about 2% w/w of the composition to about 20% w/w of the composition, about 5% w/w of the composition to about 10% w/w of the composition, about 5% w/w of the composition to about 20% w/w of the composition, or about 10% w/w of the composition to about 20% w/w of the composition. In some embodiments, the non-ionic surfactant is about 0.05% w/w of the composition, about 0.1% w/w of the composition, about 0.5% w/w of the composition, about 1% w/w of the composition, about 2% w/w of the composition, about 5% w/w of the composition, about 10% w/w of the composition, or about 20% w/w of the composition.

In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the non-ionic surfactant comprises Polysorbate 20, Poloxamer 407, Tyloxapol, or cremophor. In some embodiments, the non-ionic surfactant is Polysorbate 20. In some embodiments, the non-ionic surfactant is Poloxamer 407. In some embodiments, the non-ionic surfactant is Tyloxapol. In some embodiments, the non-ionic surfactant is cremophor. The non-ionic surfactants described herein can be present within any one of the ranges (e.g., percent w/w) described herein, a specific value that falls within the described ranges.

In some embodiments, the composition further comprises cosolvents (e.g., between 0.5 and 50% w/w), such as N,N-Dimethylacetamide, ethanol, PEG-400, propylene glycol, dimethylsulfoxide (DMSO); oils, or cyclodextrins may be added to a pharmaceutical preparation. In some embodiments, the composition further comprises a tonicity-adjusting agent. In some embodiments, the composition is an isotonic solution. In some embodiments, the tonicity-adjusting agent is mannitol, sorbitol, glucose or trehalose, or an inorganic salt such as sodium chloride. In some embodiments, the composition comprises mannitol. In some embodiments, the composition comprises sorbitol. In some embodiments, the composition comprises glucose or trehalose. In some embodiments, the composition comprises an inorganic salt. In some embodiments, the tonicity-adjusting agent is present at an amount suitable to bring the tonicity of the composition into the 250-400 mOsm/L range. In some embodiments, the non-ionic surfactant is about 1% w/w of the composition to about 10% w/w of the composition. In some embodiments, the non-ionic surfactant is at least about 1% w/w of the composition. In some embodiments, the non-ionic surfactant is at most about 10% w/w of the composition. In some embodiments, the non-ionic surfactant is about 1% w/w of the composition to about 2% w/w of the composition, about 1% w/w of the composition to about 3% w/w of the composition, about 1% w/w of the composition to about 4% w/w of the composition, about 1% w/w of the composition to about 5% w/w of the composition, about 1% w/w of the composition to about 10% w/w of the composition, about 2% w/w of the composition to about 3% w/w of the composition, about 2% w/w of the composition to about 4% w/w of the composition, about 2% w/w of the composition to about 5% w/w of the composition, about 2% w/w of the composition to about 10% w/w of the composition, about 3% w/w of the composition to about 4% w/w of the composition, about 3% w/w of the composition to about 5% w/w of the composition, about 3% w/w of the composition to about 10% w/w of the composition, about 4% w/w of the composition to about 5% w/w of the composition, about 4% w/w of the composition to about 10% w/w of the composition, or about 5% w/w of the composition to about 10% w/w of the composition. In some embodiments, the non-ionic surfactant is about 1% w/w of the composition, about 2% w/w of the composition, about 3% w/w of the composition, about 4% w/w of the composition, about 5% w/w of the composition, or about 10% w/w of the composition.

In some embodiments, the composition comprises a buffering agent. In some embodiments, the buffering agent is an acidifying agent. In some embodiments, the acidifying agent is an acetate buffer at pH 4.5. In some embodiments, the concentration acetate buffer pH 4.5 is about 10 millimolar (mM). Generally, the pH may be controlled by an appropriate buffer suitable for injection into the eye, for example the pH of the composition can be in the 3.0-7.5 range or 3.5-4.5 range.

As described herein, the composition can comprise one or more excipients. Accordingly, in some embodiments, the composition comprises a non-ionic surfactant, a tonicity-adjusting agent, and a buffering agent, in combination with the peptide. Any of the described excipients can be combined within the amounts and/or ranges described.

### Dosages and Dosing Regimens

In an embodiment, the compositions described herein comprise an amount of the peptide suitable to inhibit Fas-mediated inflammation and/or treat, inhibit, reduce, and/or prevent retinal degeneration or the symptoms thereof in an eye. As used herein, a dose or dosage includes and/or refers to the amount of therapeutic agent, such as the peptides described, in a composition (e.g., a composition for administering to an eye). A dose can refer to either (i) the peptide (parent compound) or the pharmaceutically-acceptable salt thereof. In some embodiments, the amount of the peptide in the composition (i.e., pharmaceutical composition) that is suitable for the methods described herein (e.g., treating retinal degeneration) ranges from 5 micrograms (ug) to 10,000 ug. The dosing forms comprising the compositions described herein are generally administered to the vitreous humor of an eye and can be further formulated for injection into the eye (e.g., intravitreal injection). In some embodiments, the amount of the peptide (e.g., a peptide comprising the amino acid sequence HHIYLGAVNYIY) or a pharmaceutically-acceptable salt thereof that is suitable for the methods described herein ranges from 5 ug to 10,000 ug. In some embodiments, the amount of the peptide comprising the amino acid sequence HHIYLGAVNYIY or variant sequence thereof, or a pharmaceutically-acceptable salt of the peptide that is suitable for the methods described herein ranges from 5 ug to 10,000 ug. In some embodiments, the amount of the peptide having the structure of Formula I or a pharmaceutically-acceptable salt thereof that is suitable for the methods described herein ranges from 5 ug to 10,000 ug. In some embodiments, the amount of the peptide having the structure of Formula III or a pharmaceutically-acceptable salt thereof that is suitable for the methods described herein ranges from 5 ug to 10,000 ug.

In some embodiments, the composition comprises about 5 ug of the peptide to about 10,000 ug of the peptide. In some embodiments, the composition comprises at least about 5 ug of the peptide. In some embodiments, the composition comprises at most about 10,000 ug of the peptide. In some embodiments, the composition comprises about 5 ug of the peptide to about 25 ug of the peptide, about 5 ug of the peptide to about 50 ug of the peptide, about 5 ug of the peptide to about 100 ug of the peptide, about 5 ug of the peptide to about 200 ug of the peptide, about 5 ug of the peptide to about 500 ug of the peptide, about 5 ug of the peptide to about 1,000 ug of the peptide, about 5 ug of the peptide to about 2,500 ug of the peptide, about 5 ug of the peptide to about 5,000 ug of the peptide, about 5 ug of the peptide to about 10,000 ug of the peptide, about 25 ug of the peptide to about 50 ug of the peptide, about 25 ug of the peptide to about 100 ug of the peptide, about 25 ug of the peptide to about 200 ug of the peptide, about 25 ug of the peptide to about 500 ug of the peptide, about 25 ug of the peptide to about 1,000 ug of the peptide, about 25 ug of the peptide to about 2,500 ug of the peptide, about 25 ug of the peptide to about 5,000 ug of the peptide, about 25 ug of the peptide to about 10,000 ug of the peptide, about 50 ug of the peptide to about 100 ug of the peptide, about 50 ug of the peptide to about 200 ug of the peptide, about 50 ug of the peptide to about 500 ug of the peptide, about 50 ug of the peptide to about 1,000 ug of the peptide, about 50 ug of the peptide to about 2,500 ug of the peptide, about 50 ug of the peptide to about 5,000 ug of the peptide, about 50 ug of the peptide to about 10,000 ug of the peptide, about 100 ug of the peptide to about 200 ug of the peptide, about 100 ug of the peptide to about 500 ug of the peptide, about 100 ug of the peptide to about 1,000 ug of the peptide, about 100 ug of the peptide to about 2,500 ug of the peptide, about 100 ug of the peptide to about 5,000 ug of the peptide, about 100 ug of the peptide to about 10,000 ug of the peptide, about 200 ug of the peptide to about 500 ug of the peptide, about 200 ug of the peptide to about 1,000 ug of the peptide, about 200 ug of the peptide to about 2,500 ug of the peptide, about 200 ug of the peptide to about 5,000 ug of the peptide, about 200 ug of the peptide to about 10,000 ug of the peptide, about 500 ug of the peptide to about 1,000 ug of the peptide, about 500 ug of the peptide to about 2,500 ug of the peptide, about 500 ug of the peptide to about 5,000 ug of the peptide, about 500 ug of the peptide to about 10,000 ug of the peptide, about 1,000 ug of the peptide to about 2,500 ug of the peptide, about 1,000 ug of the peptide to about 5,000 ug of the peptide, about 1,000 ug of the peptide to about 10,000 ug of the peptide, about 2,500 ug of the peptide to about 5,000 ug of the peptide, about 2,500 ug of the peptide to about 10,000 ug of the peptide, or about 5,000 ug of the peptide to about 10,000 ug of the peptide. In some embodiments, the composition comprises about 5 ug of the peptide, about 25 ug of the peptide, about 50 ug of the peptide, about 100 ug of the peptide, about 200 ug of the peptide, about 500 ug of the peptide, about 1,000 ug of the peptide, about 2,500 ug of the peptide, about 5,000 ug of the peptide, or about 10,000 ug of the peptide.

In some embodiments, a dose comprises about 5 ug of a pharmaceutically-acceptable salt of the peptide to about 10,000 ug of a pharmaceutically-acceptable salt of the peptide. In some embodiments, a dose comprises at least about 5 ug of a pharmaceutically-acceptable salt of the peptide. In some embodiments, a dose comprises at most about 10,000 ug of a pharmaceutically-acceptable salt of the peptide. In some embodiments, a dose comprises about 5 ug of a pharmaceutically-acceptable salt of the peptide to about 100 ug of a pharmaceutically-acceptable salt of the peptide, about 5 ug of a pharmaceutically-acceptable salt of the peptide to about 200 ug of a pharmaceutically-acceptable salt of the peptide, about 5 ug of a pharmaceutically-acceptable salt of the peptide to about 500 ug of a pharmaceutically-acceptable salt of the peptide, about 5 ug of a pharmaceutically-acceptable salt of the peptide to about 1,000 ug of a pharmaceutically-acceptable salt of the peptide, about 5 ug of a pharmaceutically-acceptable salt of the peptide to about 2,500 ug of a pharmaceutically-acceptable salt of the peptide, about 5 ug of a pharmaceutically-acceptable salt of the peptide to about 5,000 ug of a pharmaceutically-acceptable salt of the peptide, about 5 ug of a pharmaceutically-acceptable salt of the peptide to about 10,000 ug of a pharmaceutically-acceptable salt of the peptide, about 100 ug of a pharmaceutically-acceptable salt of the peptide to about 200 ug of a pharmaceutically-acceptable salt of the peptide, about 100 ug of a pharmaceutically-acceptable salt of the peptide to about 500 ug of a pharmaceutically-acceptable salt of the peptide, about 100 ug of a pharmaceutically-acceptable salt of the peptide to about 1,000 ug of a pharmaceutically-acceptable salt of the peptide, about 100 ug of a pharmaceutically-acceptable salt of the peptide to about 2,500 ug of a pharmaceutically-acceptable salt of the peptide, about 100 ug of a pharmaceutically-acceptable salt of the peptide to about 5,000 ug of a pharmaceutically-acceptable salt of the peptide, about 100 ug of a pharmaceutically-acceptable salt of the peptide to about 10,000 ug of a pharmaceutically-acceptable salt of the peptide, about 200 ug of a pharmaceutically-acceptable salt of the peptide to about 500 ug of a pharmaceutically-acceptable salt of the peptide, about 200 ug of a pharmaceutically-acceptable salt of the peptide to about 1,000 ug of a pharmaceutically-acceptable salt of the peptide, about 200 ug of a pharmaceutically-acceptable salt of the peptide to about 2,500 ug of a pharmaceutically-acceptable salt of the peptide, about 200 ug of a pharmaceutically-acceptable salt of the peptide to about 5,000 ug of a pharmaceutically-acceptable salt of the peptide, about 200 ug of a pharmaceutically-acceptable salt of the peptide to about 10,000 ug of a pharmaceutically-acceptable salt of the peptide, about 500 ug of a pharmaceutically-acceptable salt of the peptide to about 1,000 ug of a pharmaceutically-acceptable salt of the peptide, about 500 ug of a pharmaceutically-acceptable salt of the peptide to about 2,500 ug of a pharmaceutically-acceptable salt of the peptide, about 500 ug of a pharmaceutically-acceptable salt of the peptide to about 5,000 ug of a pharmaceutically-acceptable salt of the peptide, about 500 ug of a pharmaceutically-acceptable salt of the peptide to about 10,000 ug of a pharmaceutically-acceptable salt of the peptide, about 1,000 ug of a pharmaceutically-acceptable salt of the peptide to about 2,500 ug of a pharmaceutically-acceptable salt of the peptide, about 1,000 ug of a pharmaceutically-acceptable salt of the peptide to about 5,000 ug of a pharmaceutically-acceptable salt of the peptide, about 1,000 ug of a pharmaceutically-acceptable salt of the peptide to about 10,000 ug of a pharmaceutically-acceptable salt of the peptide, about 2,500 ug of a pharmaceutically-acceptable salt of the peptide to about 5,000 ug of a pharmaceutically-acceptable salt of the peptide, about 2,500 ug of a pharmaceutically-acceptable salt of the peptide to about 10,000 ug of a pharmaceutically-acceptable salt of the peptide, or about 5,000 ug of a pharmaceutically-acceptable salt of the peptide to about 10,000 ug of a pharmaceutically-acceptable salt of the peptide. In some embodiments, a dose comprises about 5 ug of a pharmaceutically-acceptable salt of the peptide, about 100 ug of a pharmaceutically-acceptable salt of the peptide, about 200 ug of a pharmaceutically-acceptable salt of the peptide, about 500 ug of a pharmaceutically-acceptable salt of the peptide, about 1,000 ug of a pharmaceutically-acceptable salt of the peptide, about 2,500 ug of a pharmaceutically-acceptable salt of the peptide, about 5,000 ug of a pharmaceutically-acceptable salt of the peptide, or about 10,000 ug of a pharmaceutically-acceptable salt of the peptide.

In some embodiments, the amount of the peptide comprising the amino acid sequence HHIYLGAVNYIY or variant sequence thereof, or a pharmaceutically-acceptable salt of the peptide that is suitable for the methods described herein ranges from 5 ug to 300 ug. In some embodiments, the amount of the peptide having the structure of Formula I or a pharmaceutically-acceptable salt thereof that is suitable for the methods described herein ranges from 5 ug to 300 ug. In some embodiments, the amount of the peptide having the structure of Formula III or a pharmaceutically-acceptable salt thereof that is suitable for the methods described herein ranges from 5 ug to 300 ug.

In some embodiments, the composition comprises about 5 ug of the peptide to about 300 ug of the peptide. In some embodiments, the composition comprises at least about 5 ug of the peptide. In some embodiments, the composition comprises at most about 300 ug of the peptide. In some embodiments, the composition comprises about 5 ug of the peptide to about 10 ug of the peptide, about 5 ug of the peptide to about 25 ug of the peptide, about 5 ug of the peptide to about 50 ug of the peptide, about 5 ug of the peptide to about 75 ug of the peptide, about 5 ug of the peptide to about 100 ug of the peptide, about 5 ug of the peptide to about 150 ug of the peptide, about 5 ug of the peptide to about 200 ug of the peptide, about 5 ug of the peptide to about 250 ug of the peptide, about 5 ug of the peptide to about 300 ug of the peptide, about 10 ug of the peptide to about 25 ug of the peptide, about 10 ug of the peptide to about 50 ug of the peptide, about 10 ug of the peptide to about 75 ug of the peptide, about 10 ug of the peptide to about 100 ug of the peptide, about 10 ug of the peptide to about 150 ug of the peptide, about 10 ug of the peptide to about 200 ug of the peptide, about 10 ug of the peptide to about 250 ug of the peptide, about 10 ug of the peptide to about 300 ug of the peptide, about 25 ug of the peptide to about 50 ug of the peptide, about 25 ug of the peptide to about 75 ug of the peptide, about 25 ug of the peptide to about 100 ug of the peptide, about 25 ug of the peptide to about 150 ug of the peptide, about 25 ug of the peptide to about 200 ug of the peptide, about 25 ug of the peptide to about 250 ug of the peptide, about 25 ug of the peptide to about 300 ug of the peptide, about 50 ug of the peptide to about 75 ug of the peptide, about 50 ug of the peptide to about 100 ug of the peptide, about 50 ug of the peptide to about 150 ug of the peptide, about 50 ug of the peptide to about 200 ug of the peptide, about 50 ug of the peptide to about 250 ug of the peptide, about 50 ug of the peptide to about 300 ug of the peptide, about 75 ug of the peptide to about 100 ug of the peptide, about 75 ug of the peptide to about 150 ug of the peptide, about 75 ug of the peptide to about 200 ug of the peptide, about 75 ug of the peptide to about 250 ug of the peptide, about 75 ug of the peptide to about 300 ug of the peptide, about 100 ug of the peptide to about 150 ug of the peptide, about 100 ug of the peptide to about 200 ug of the peptide, about 100 ug of the peptide to about 250 ug of the peptide, about 100 ug of the peptide to about 300 ug of the peptide, about 150 ug of the peptide to about 200 ug of the peptide, about 150 ug of the peptide to about 250 ug of the peptide, about 150 ug of the peptide to about 300 ug of the peptide, about 200 ug of the peptide to about 250 ug of the peptide, about 200 ug of the peptide to about 300 ug of the peptide, or about 250 ug of the peptide to about 300 ug of the peptide. In some embodiments, the composition comprises about 5 ug of the peptide, about 10 ug of the peptide, about 25 ug of the peptide, about 50 ug of the peptide, about 75 ug of the peptide, about 100 ug of the peptide, about 150 ug of the peptide, about 200 ug of the peptide, about 250 ug of the peptide, or about 300 ug of the peptide.

In some embodiments, a dose comprises about 5 ug of a pharmaceutically-acceptable salt of the peptide to about 300 ug of a pharmaceutically-acceptable salt of the peptide. In some embodiments, a dose comprises at least about 5 ug of a pharmaceutically-acceptable salt of the peptide. In some embodiments, a dose comprises at most about 300 ug of a pharmaceutically-acceptable salt of the peptide. In some embodiments, a dose comprises about 5 ug of a pharmaceutically-acceptable salt of the peptide to about 25 ug of a pharmaceutically-acceptable salt of the peptide, about 5 ug of a pharmaceutically-acceptable salt of the peptide to about 50 ug of a pharmaceutically-acceptable salt of the peptide, about 5 ug of a pharmaceutically-acceptable salt of the peptide to about 100 ug of a pharmaceutically-acceptable salt of the peptide, about 5 ug of a pharmaceutically-acceptable salt of the peptide to about 150 ug of a pharmaceutically-acceptable salt of the peptide, about 5 ug of a pharmaceutically-acceptable salt of the peptide to about 200 ug of a pharmaceutically-acceptable salt of the peptide, about 5 ug of a pharmaceutically-acceptable salt of the peptide to about 250 ug of a pharmaceutically-acceptable salt of the peptide, about 5 ug of a pharmaceutically-acceptable salt of the peptide to about 300 ug of a pharmaceutically-acceptable salt of the peptide, about 25 ug of a pharmaceutically-acceptable salt of the peptide to about 50 ug of a pharmaceutically-acceptable salt of the peptide, about 25 ug of a pharmaceutically-acceptable salt of the peptide to about 100 ug of a pharmaceutically-acceptable salt of the peptide, about 25 ug of a pharmaceutically-acceptable salt of the peptide to about 150 ug of a pharmaceutically-acceptable salt of the peptide, about 25 ug of a pharmaceutically-acceptable salt of the peptide to about 200 ug of a pharmaceutically-acceptable salt of the peptide, about 25 ug of a pharmaceutically-acceptable salt of the peptide to about 250 ug of a pharmaceutically-acceptable salt of the peptide, about 25 ug of a pharmaceutically-acceptable salt of the peptide to about 300 ug of a pharmaceutically-acceptable salt of the peptide, about 50 ug of a pharmaceutically-acceptable salt of the peptide to about 100 ug of a pharmaceutically-acceptable salt of the peptide, about 50 ug of a pharmaceutically-acceptable salt of the peptide to about 150 ug of a pharmaceutically-acceptable salt of the peptide, about 50 ug of a pharmaceutically-acceptable salt of the peptide to about 200 ug of a pharmaceutically-acceptable salt of the peptide, about 50 ug of a pharmaceutically-acceptable salt of the peptide to about 250 ug of a pharmaceutically-acceptable salt of the peptide, about 50 ug of a pharmaceutically-acceptable salt of the peptide to about 300 ug of a pharmaceutically-acceptable salt of the peptide, about 100 ug of a pharmaceutically-acceptable salt of the peptide to about 150 ug of a pharmaceutically-acceptable salt of the peptide, about 100 ug of a pharmaceutically-acceptable salt of the peptide to about 200 ug of a pharmaceutically-acceptable salt of the peptide, about 100 ug of a pharmaceutically-acceptable salt of the peptide to about 250 ug of a pharmaceutically-acceptable salt of the peptide, about 100 ug of a pharmaceutically-acceptable salt of the peptide to about 300 ug of a pharmaceutically-acceptable salt of the peptide, about 150 ug of a pharmaceutically-acceptable salt of the peptide to about 200 ug of a pharmaceutically-acceptable salt of the peptide, about 150 ug of a pharmaceutically-acceptable salt of the peptide to about 250 ug of a pharmaceutically-acceptable salt of the peptide, about 150 ug of a pharmaceutically-acceptable salt of the peptide to about 300 ug of a pharmaceutically-acceptable salt of the peptide, about 200 ug of a pharmaceutically-acceptable salt of the peptide to about 250 ug of a pharmaceutically-acceptable salt of the peptide, about 200 ug of a pharmaceutically-acceptable salt of the peptide to about 300 ug of a pharmaceutically-acceptable salt of the peptide, or about 250 ug of a pharmaceutically-acceptable salt of the peptide to about 300 ug of a pharmaceutically-acceptable salt of the peptide. In some embodiments, a dose comprises about 5 ug of a pharmaceutically-acceptable salt of the peptide, about 25 ug of a pharmaceutically-acceptable salt of the peptide, about 50 ug of a pharmaceutically-acceptable salt of the peptide, about 100 ug of a pharmaceutically-acceptable salt of the peptide, about 150 ug of a pharmaceutically-acceptable salt of the peptide, about 200 ug of a pharmaceutically-acceptable salt of the peptide, about 250 ug of a pharmaceutically-acceptable salt of the peptide, or about 300 ug of a pharmaceutically-acceptable salt of the peptide.

The concentration of the peptide within the composition can be adjusted in a manner suitable for ocular administration. In some embodiments, the concentration of the peptide within the composition ranges from about 0.1 milligrams per milliliter (mg/mL) to about 5 mg/mL. In some embodiments, the concentration of the peptide within the composition ranges from about 0.1 milligrams per milliliter (mg/mL) to about 10 mg/mL. In some embodiments, the concentration of the peptide within the composition ranges from about 0.1 milligrams per milliliter (mg/mL) to about 100 mg/mL. In some embodiments, the concentration of the peptide (e.g., a peptide comprising the amino acid sequence HHIYLGAVNYIY) or a pharmaceutically-acceptable salt thereof ranges from about 0.1 mg/mL to about 5 mg/mL. In some embodiments, the concentration of the peptide comprising the amino acid sequence HHIYLGAVNYIY or variant sequence thereof, or a pharmaceutically-acceptable salt of the peptide ranges from about 0.1 milligrams per milliliter (mg/mL) to about 5 mg/mL. In some embodiments, the concentration of the peptide having the structure of Formula I or a pharmaceutically-acceptable salt thereof ranges from about 0.1 mg/mL to about 5 mg/mL. In some embodiments, the concentration of the peptide having the structure of Formula III or a pharmaceutically-acceptable salt thereof ranges from about 0.1 mg/mL to about 5 mg/mL.

In some embodiments, the concentration of the peptide within the composition is about 0.1 mg/mL to about 5 mg/mL. In some embodiments, the concentration of the peptide within the composition is at least about 0.1 mg/mL. In some embodiments, the concentration of the peptide within the composition is at most about 5 mg/mL. In some embodiments, the concentration of the peptide within the composition is about 0.1 mg/mL to about 0.5 mg/mL, about 0.1 mg/mL to about 1 mg/mL, about 0.1 mg/mL to about 1.5 mg/mL, about 0.1 mg/mL to about 2 mg/mL, about 0.1 mg/mL to about 2.5 mg/mL, about 0.1 mg/mL to about 3 mg/mL, about 0.1 mg/mL to about 4 mg/mL, about 0.1 mg/mL to about 5 mg/mL, about 0.5 mg/mL to about 1 mg/mL, about 0.5 mg/mL to about 1.5 mg/mL, about 0.5 mg/mL to about 2 mg/mL, about 0.5 mg/mL to about 2.5 mg/mL, about 0.5 mg/mL to about 3 mg/mL, about 0.5 mg/mL to about 4 mg/mL, about 0.5 mg/mL to about 5 mg/mL, about 1 mg/mL to about 1.5 mg/mL, about 1 mg/mL to about 2 mg/mL, about 1 mg/mL to about 2.5 mg/mL, about 1 mg/mL to about 3 mg/mL, about 1 mg/mL to about 4 mg/mL, about 1 mg/mL to about 5 mg/mL, about 1.5 mg/mL to about 2 mg/mL, about 1.5 mg/mL to about 2.5 mg/mL, about 1.5 mg/mL to about 3 mg/mL, about 1.5 mg/mL to about 4 mg/mL, about 1.5 mg/mL to about 5 mg/mL, about 2 mg/mL to about 2.5 mg/mL, about 2 mg/mL to about 3 mg/mL, about 2 mg/mL to about 4 mg/mL, about 2 mg/mL to about 5 mg/mL, about 2.5 mg/mL to about 3 mg/mL, about 2.5 mg/mL to about 4 mg/mL, about 2.5 mg/mL to about 5 mg/mL, about 3 mg/mL to about 4 mg/mL, about 3 mg/mL to about 5 mg/mL, or about 4 mg/mL to about 5 mg/mL. In some embodiments, the concentration of the peptide within the composition is about 0.1 mg/mL, about 0.5 mg/mL, about 1 mg/mL, about 1.5 mg/mL, about 2 mg/mL, about 2.5 mg/mL, about 3 mg/mL, about 4 mg/mL, or about 5 mg/mL.

### Pharmacokinetics

As contemplated herein, the compositions described herein are administered to an eye of an individual in need thereof. Administration to an eye (i.e., “ocular application” or “ocular administration”) includes subconjunctival, intravitreal, retrobulbar, intracameral administration subretinal, or suprachoroidal. In some embodiments, ocular administration comprises subconjunctival, intravitreal, retrobulbar, or intracameral administration. In some embodiments, ocular administration comprises intravitreal administration. In some embodiments, ocular administration comprises subconjunctival administration. In some embodiments, ocular administration comprises retrobulbar administration. In some embodiments, ocular administration comprises intracameral administration.

In some embodiments, the dosing forms comprising the compositions described herein are generally administered to the vitreous humor of an eye. In some embodiments, the half-life of the peptide (e.g., a peptide comprising the amino acid sequence HHIYLGAVNYIY) or a pharmaceutically-acceptable salt thereof in the vitreous humor is greater than about 30 days to greater than about 275 days. In some embodiments, the peptide comprising the amino acid sequence HHIYLGAVNYIY or variant sequence thereof, or a pharmaceutically-acceptable salt of the peptide has a half-life in the vitreous humor that is greater than about 30 days to greater than about 275 days. In some embodiments, the peptide having the structure of Formula I or a pharmaceutically-acceptable salt thereof has a half-life in the vitreous humor that is greater than is greater than about 30 days to greater than about 275 days. In some embodiments, the peptide having the structure of Formula III or a pharmaceutically-acceptable salt thereof has a half-life in the vitreous humor that is greater than is greater than about 30 days to greater than about 275 days. In some embodiments, the peptide comprising the amino acid sequence HHIYLGAVNYIY or variant sequence thereof, or a pharmaceutically-acceptable salt of the peptide has a half-life in the vitreous humor that is greater than about 14 days to greater than about 275 days. In some embodiments, the peptide having the structure of Formula I or a pharmaceutically-acceptable salt thereof has a half-life in the vitreous humor that is greater than is greater than about 14 days to greater than about 275 days. In some embodiments, the peptide having the structure of Formula III or a pharmaceutically-acceptable salt thereof has a half-life in the vitreous humor that is greater than is greater than about 14 days to greater than about 275 days.

In some embodiments, the half-life of the peptide is greater than about 14 days in the eye. In some embodiments, the half-life of the peptide is greater than about 30 days in the eye. In some embodiments, the half-life of the peptide is greater than about 60 days in the eye. In some embodiments, the half-life of the peptide is greater than about 90 days in the eye. In some embodiments, the half-life of the peptide is greater than about 120 days in the eye. In some embodiments, the half-life of the peptide is greater than about 150 days in the eye. In some embodiments, the half-life of the peptide is greater than about 180 days in the eye. In some embodiments, the half-life of the peptide is greater than about 210 days in the eye. In some embodiments, the half-life of the peptide is greater than about 240 days in the eye. In some embodiments, the half-life of the peptide is greater than about 270 days in the eye.

In some embodiments, the half-life of the peptide is greater than about 14 days in the vitreous humor. In some embodiments, the half-life of the peptide is greater than about 30 days in the vitreous humor. In some embodiments, the half-life of the peptide is greater than about 60 days in the vitreous humor. In some embodiments, the half-life of the peptide is greater than about 90 days in the vitreous humor. In some embodiments, the half-life of the peptide is greater than about 120 days in the vitreous humor. In some embodiments, the half-life of the peptide is greater than about 150 days in the vitreous humor. In some embodiments, the half-life of the peptide is greater than about 180 days in the vitreous humor. In some embodiments, the half-life of the peptide is greater than about 210 days in the vitreous humor. In some embodiments, the half-life of the peptide is greater than about 240 days in the vitreous humor. In some embodiments, the half-life of the peptide is greater than about 270 days in the vitreous humor.

Determining the amount of the peptide in the vitreous humor generally requires collecting all of the vitreous fluid or a substantial portion thereof from an eye or sacrificing the eye in order to sample the vitreous humor. In some embodiments, collecting all of the vitreous fluid or a substantial portion thereof in a human eye, or sacrificing an eye is not feasible for maintaining the health of an eye in a human. Accordingly, in some embodiments, the half-life of the peptide in a human eye is determined by measuring and/or extrapolating from a half-life of the peptide in the eye of a mammal. In some embodiments, the mammal is a rabbit. In some embodiments, the mammal is a pig (e.g., minipig). In some embodiments, the mammal is a monkey. Various methods of detecting the presence of a drug are also suitable for detecting the peptide. For example, methods suitable for detecting the peptide include performing mass spectrometry (e.g., liquid chromatography-mass spectrometry (LC-MS) or high-performance LC-MS (HPLC-MS)) on a sample from the vitreous humor.

### Methods

Provided herein are methods that are advantageous for use in improving visual function in an individual having an ocular disease and/or disorder. In some embodiments, the ocular disease and/or disorder comprises a loss in visual function (e.g., best corrected visual acuity, field of vision, contrast sensitivity, binocular function, low luminance acuity, low contrast acuity, color vision, perimetry, threshold sensitivity, reading speed, and/or light/dark adaptation). In some embodiments, the ocular disease and/or disorder comprises photoreceptor cell death. In some embodiments, the ocular disease and/or disorder comprises retinal pigment epithelial cell death. In some embodiments, the ocular disease and/or disorder comprises degeneration of the macula. In some embodiments, the ocular disease and/or disorder comprises inflammation within the eye and/or retinal tissue (e.g., as measured by pro-inflammatory markers within a sample from the vitreous).

Described and provided herein are methods of improving visual function (e.g., BCVA) in an eye of an individual having an ocular disease or disorder, comprising: administering a Fas inhibitor to the eye of the individual. Further described and provided herein are methods of improving visual function (e.g., BCVA) in an eye of an individual having an ocular disease or disorder, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual. Further described and provided herein are methods of improving visual function (e.g., BCVA) in an eye of an individual having an ocular disease or disorder, comprising: administering a peptide (e.g., a composition comprising the peptide) having the structure of Formula I or a pharmaceutically-acceptable salt thereof to the eye of the individual. Described and provided herein are methods of improving visual function (e.g., BCVA) in an eye of an individual having an ocular disease or disorder, comprising: administering a peptide (e.g., a composition comprising the peptide) having the structure of Formula III or a pharmaceutically-acceptable salt thereof to the eye of the individual. In some embodiments, the ocular disease or disorder comprises a loss in visual function. In some embodiments, the ocular disease or disorder comprises photoreceptor cell death or a symptom thereof (e.g., a loss in visual function). In some embodiments, the ocular disease or disorder comprises a loss in visual function. In some embodiments, the ocular disease or disorder comprises photoreceptor cell death or a symptom thereof (e.g., a loss in visual function). In some embodiments, the ocular disease or disorder comprises retinal pigment epithelial cell death or a symptom thereof (e.g., a loss in visual function). In some embodiments, the ocular disease or disorder comprises degeneration of the macula. In some embodiments, the method comprises improving visual function without performing surgery. In some embodiments, the surgery is performed after administering the peptide or the variant sequence thereof, or the pharmaceutically-acceptable salt of the peptide. In some embodiments, surgery comprises retinal reattachment surgery. In some embodiments, surgery comprises a vitrectomy.

Further described and provided herein are methods of treating a loss in visual function (e.g., BCVA) in an eye of an individual having macular degeneration, comprising: administering a Fas inhibitor to the eye of the individual. Also described and provided are methods of treating a loss in visual function (e.g., BCVA) in an eye of an individual having macular degeneration, comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual. Further described and provided herein are methods treating a loss in visual function (e.g., BCVA) in an eye of an individual having macular degeneration, comprising: administering a peptide (e.g., a composition comprising the peptide) having the structure of Formula I or a pharmaceutically-acceptable salt thereof to the eye of the individual. Described and provided herein are methods of treating a loss in visual function (e.g., BCVA) in an eye of an individual having macular degeneration, comprising: administering a peptide (e.g., a composition comprising the peptide) having the structure of Formula III or a pharmaceutically-acceptable salt thereof to the eye of the individual. In some embodiments, the macular degeneration comprises age-related macular degeneration. In some embodiments, the macular degeneration comprises geographic atrophy.

Also described and provided herein are methods of treating macular degeneration in an eye of an individual having geographical atrophy (e.g., AMD characterized by geographical atrophy), comprising: administering a Fas inhibitor to the eye of the individual. Further described and provided are methods of treating macular degeneration in an eye of an individual having geographical atrophy (e.g., AMD characterized by geographical atrophy), comprising: administering a peptide (e.g., a composition comprising the peptide) comprising an amino acid sequence HHIYLGAVNYIY or a variant sequence thereof, or a pharmaceutically-acceptable salt of the peptide, to the eye of the individual. Further described and provided herein are methods of treating macular degeneration in an eye of an individual having geographical atrophy (e.g., AMD characterized by geographical atrophy), comprising: administering a peptide (e.g., a composition comprising the peptide) having the structure of Formula I or a pharmaceutically-acceptable salt thereof to the eye of the individual. Described and provided herein are methods of treating macular degeneration in an eye of an individual having geographical atrophy (e.g., AMD characterized by geographical atrophy), comprising: administering a peptide (e.g., a composition comprising the peptide) having the structure of Formula III or a pharmaceutically-acceptable salt thereof to the eye of the individual. In some embodiments, the macular degeneration comprises age-related macular degeneration.

In some embodiments, treating visual function comprises improving visual function as compared to a baseline visual function prior to administering the peptide or the variant sequence thereof, or the pharmaceutically-acceptable salt of the peptide. In some embodiments, visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation. In some embodiments, visual function comprises best corrected visual acuity (e.g., increase in visual acuity from 20/200 to 20/100, or an increase in visual acuity from 20/50 to 20/40, and/or an increase in visual acuity from measured in a reduction in logMAR, etc.). In some embodiments, visual function comprises field of vision. In some embodiments, visual function comprises contrast sensitivity. In some embodiments, visual function comprises binocular function. In some embodiments, visual function comprises low luminance acuity. In some embodiments, visual function comprises low contrast acuity. In some embodiments, visual function comprises color vision. In some embodiments, visual function comprises perimetry. In some embodiments, visual function comprises threshold sensitivity. In some embodiment, visual function comprises reading speed. In some embodiments, visual function comprises light/dark adaptation. In some embodiments, improving visual function comprises improving visual function as compared to a baseline visual function prior to the administering peptide or the variant sequence thereof, or the pharmaceutically-acceptable salt of the peptide.

In some embodiments, the methods described herein include a methods of treating vision loss associated with inflammation (e.g., Fas-mediated inflammation) in an eye. In some embodiments, retinal inflammation can be determined by observing the symptoms associated with inflammation in the eye (e.g., loss of and/or decrease in visual acuity, central vision loss, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-lb, IP-10, IL-18, MIP-la, IL-6, GFAP, MIP2, MCP-1, or MIP-lb); a Fas-mediated complement-related molecules (complement component 3 (C3) or complement component 1 q (C1q)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-la), and CCL4 (MIP-lb)); toll-like receptor 4 (TLR4); interleukin cytokines (e.g., IL-lb, IL-18, and IL-6); TNF superfamily cytokines (e.g., TNFa); or GFAP.

In some embodiments, the peptide or the variant sequence thereof, or the pharmaceutically-acceptable salt of the peptide is administered via intravitreal injection, intracameral injection, injection into the suprachoroidal space, injection in the subtenons space, subconjunctival injection, retrobulbar injection, peribulbar injection, microneedle injection, subretinal injection, or subretinal infusion. In certain embodiments, the peptide or the variant sequence thereof, or the pharmaceutically-acceptable salt of the peptide is administered via intravitreal injection. In certain embodiments, the peptide or the variant sequence thereof, or the pharmaceutically-acceptable salt of the peptide is administered via intracameral injection. In certain embodiments, the peptide or the variant sequence thereof, or the pharmaceutically-acceptable salt of the peptide is administered via injection into the suprachoroidal space. In certain embodiments, the peptide or the variant sequence thereof, or the pharmaceutically-acceptable salt of the peptide is administered via injection in the subtenons space. In certain embodiments, the peptide or the variant sequence thereof, or the pharmaceutically-acceptable salt of the peptide is administered via subconjunctival injection. In certain embodiments, the peptide or the variant sequence thereof, or the pharmaceutically-acceptable salt of the peptide is administered via retrobulbar injection. In certain embodiments, the peptide or the variant sequence thereof, or the pharmaceutically-acceptable salt of the peptide is administered via peribulbar injection. In certain embodiments, the peptide or the variant sequence thereof, or the pharmaceutically-acceptable salt of the peptide is administered via microneedle injection. In certain embodiments, the peptide or the variant sequence thereof, or the pharmaceutically-acceptable salt of the peptide is administered via subretinal injection. In certain embodiments, the peptide or the variant sequence thereof, or the pharmaceutically-acceptable salt of the peptide is administered via subretinal infusion.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, comprises a modified amino acid or a non-natural amino acid. In some embodiments, the peptide comprises an amidated C-terminus. In some embodiments, the peptide has the structure of Formula I, or a pharmaceutically-acceptable salt thereof. In some embodiments, the peptide has the structure of Formula III, or a pharmaceutically-acceptable salt thereof.

In some embodiments, the pharmaceutically-acceptable salt of the peptide is administered. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, the peptide or the variant sequence thereof, or the pharmaceutically-acceptable salt of the peptide is formulated in a composition (e.g., the pharmaceutical compositions described herein).

In some embodiments, about 5-1,000 ug of the peptide or the variant sequence thereof, or the pharmaceutically-acceptable salt of the peptide is administered. In some embodiments, about 5-500 ug of the peptide or the variant sequence thereof, or the pharmaceutically-acceptable salt of the peptide is administered. In some embodiments, about 5-200 ug of the peptide or the variant sequence thereof, or the pharmaceutically-acceptable salt of the peptide is administered. In some embodiments, about 5-100 ug of the peptide or the variant sequence thereof, or the pharmaceutically-acceptable salt of the peptide is administered. In some embodiments, about 5-50 ug of the peptide or the variant sequence thereof, or the pharmaceutically-acceptable salt of the peptide is administered. In some embodiments, about 50-500 ug of the peptide or the variant sequence thereof, or the pharmaceutically-acceptable salt of the peptide is administered. In some embodiments, about 50-200 ug of the peptide or the variant sequence thereof, or the pharmaceutically-acceptable salt of the peptide is administered. In some embodiments, about 50-100 ug of the peptide or the variant sequence thereof, or the pharmaceutically-acceptable salt of the peptide is administered.

In some embodiments, about 5 ug of the peptide or the variant sequence thereof, or the pharmaceutically-acceptable salt of the peptide is administered. In some embodiments, about 25 ug of the peptide or the variant sequence thereof, or the pharmaceutically-acceptable salt of the peptide is administered. In some embodiments, about 50 ug of the peptide or the variant sequence thereof, or the pharmaceutically-acceptable salt of the peptide is administered. In some embodiments, 100 ug of the peptide or the variant sequence thereof, or the pharmaceutically-acceptable salt of the peptide is administered. In some embodiments, about 200 ug of the peptide or the variant sequence thereof, or the pharmaceutically-acceptable salt of the peptide is administered. In some embodiments, about 500 ug of the peptide or the variant sequence thereof, or the pharmaceutically-acceptable salt of the peptide is administered. In some embodiments, about 1000 ug of the peptide or the variant sequence thereof, or the pharmaceutically-acceptable salt of the peptide is administered.

In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

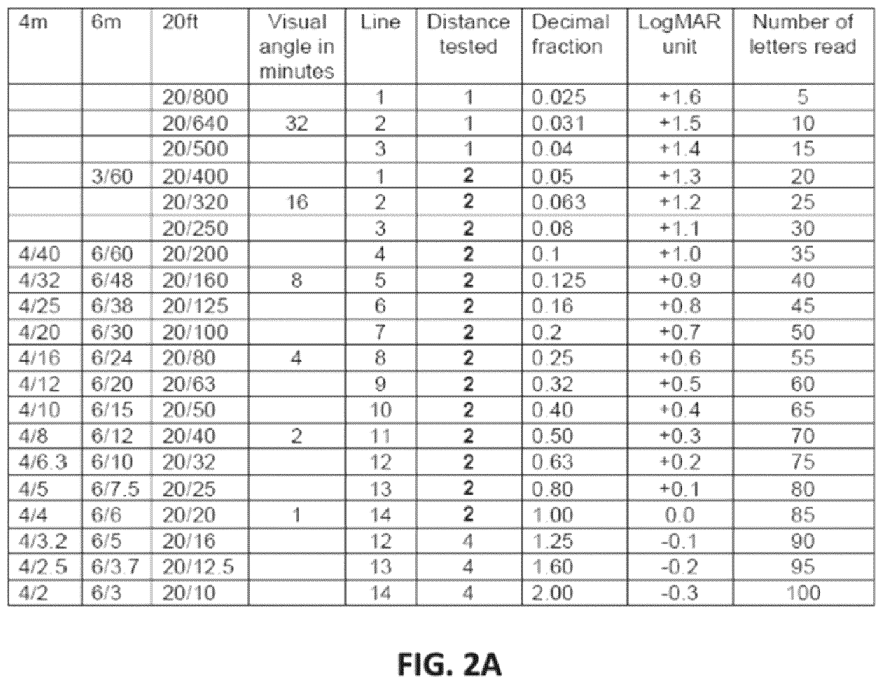

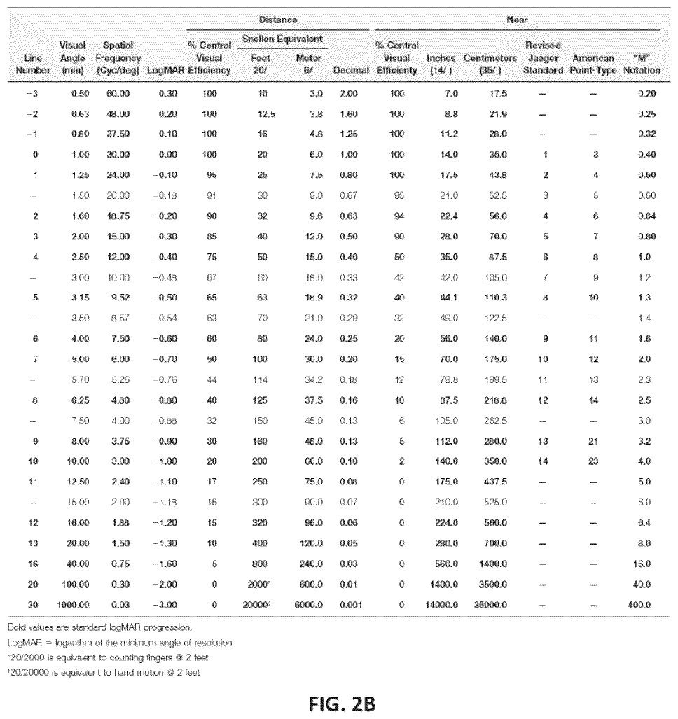

As used herein, the term “best corrected visual acuity” or “BCVA”, generally refers to the minimum angle of resolution subtended by a certain number of arc minutes. In certain instances, the subtended visual angle of an object is the angle formed by rays projecting from the eye to the top and bottom (or left and right sides) of an object. In such instances, such visual angles are used to indicate the size of the retinal image of the object (e.g., the larger the visual angle, the larger the retinal image size is). In certain instances, the visual angle is influenced by two parameters: the size of the object and the distance of the object from the eye. Bigger objects cast larger images on the retina than smaller objects. Thus, the larger the object is, the larger its visual angle will be. Closer objects cast larger images on the retina than smaller objects. Thus, the closer the object is to the eye, the larger its visual angle will be. By way of example, standard vision is thus the ability to distinguish features separated by 1 minute of arc. By way of further example, the ability to distinguish a set of bars separated by 1 arc minute is 20/20 vision, 2 arc minutes is 20/40 vision. In some embodiments, visual acuity is measured by a Snellen chart, ETDRS chart, Landolt C chart, Tumbling E chart, HOTV chart, and/or logMAR (log minimum angle of resolution). FIG. 2A-2B show an exemplary, but non-limiting, conversion chart for comparing BCVA values.

As used herein, macular degeneration generally includes and/or refers to any of a number of conditions in which the retinal macula degenerates and/or becomes dysfunctional (e.g., as a consequence of decreased growth of cells of the macula, increased death or rearrangement of the cells of the macula (e.g., RPE cells), loss of normal biological function, or a combination thereof). Macular degeneration generally, results in the loss of integrity of the histological architecture of the cells and/or extracellular matrix of the normal macula and/or the loss of function of the cells of the macula. As used herein, macular degeneration also includes and/or refers to extramacular changes that occur prior to, or following dysfunction and/or degeneration of the macula. In some embodiments, the method includes treating symptoms associated with macular degeneration (e.g., retinal degeneration, increased intraocular pressure, loss of and/or decrease in visual acuity, blurred vision, distorted vision, etc.). In some embodiments, treating comprises improving visual function (e.g., compared to a baseline visual function measured prior to treatment). In some embodiments, the macular degeneration is age-related macular degeneration. In some embodiments, the macular degeneration comprises geographic atrophy. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors

### Methods Including Surgery

Despite advances in surgical techniques for treating detachment of the neural retina (e.g., retinal reattachment surgery), functional outcomes are often poor and/or suboptimal. Even after considering such advances in surgical treatments, less than about half of all patients attain 20/40 vision, and when considering only cases where the macula is involved, only about a quarter of patients show similar outcomes. Generally, recovery of vision worsens as the time of detachment increases. This loss and potentially permanent damage to the patient's visual acuity is generally attributable to photoreceptor cell death and retinal anoxia resulting from the effects of the detached state of the retina. Further challenges in effectively treating retinal detachment also arise because, in some instances, it is not uncommon for the diagnosis of retinal detachment to be delayed because of a patient's lack of access to care or lack of knowledge about the symptoms. Additional challenges arise when considering other factors such as the time of detachment (e.g., as measurable by central vision loss), extent of detachment, size of detachment, age of the individual, and/or the inflammatory state in the eye. Generally, as such factors increase, treatment outcomes (e.g., best correct visual acuity after treatment) associated with surgery alone worsen. The methods and compositions described herein provide a solution to the challenges of treating detachment of the neural retina and ocular diseases or disorders involving detachment of the neural retina. Furthermore, in certain instances, methods and compositions described herein provide advantages to meeting such challenges over the standard of care surgery alone (e.g., surgery to reattach the retina).

Provided herein are methods that are advantageous for use in treating a patient having a detached retina (e.g., full or partially detached). In some embodiments, the methods described herein include the combination of administering a Fas inhibitor (e.g., a Fas inhibiting peptide) to an eye of an individual and performing surgery on the eye. In some embodiments, the methods described herein include a method of treating retinal detachment. In some embodiments, the method includes treating symptoms associated retinal detachment (e.g., loss of and/or decrease in visual acuity, central vision loss, blurred vision, distorted vision, etc.). As used herein, retinal detachment generally includes and refers to a condition and/or disorder of the eye in which the neural retina detaches (e.g., peels away, separates, etc.) from the underlying layer of support tissue. In some embodiments, retinal detachment refers to an instance wherein the neural retina detaches (e.g., peels away, separates, etc.) from the underlying layer of support tissue. In some embodiments, retinal detachment refers to a disease or disorder wherein the neural retina detaches (e.g., peels away, separates, etc.) from the underlying layer of support tissue. In some embodiments, types of retinal detachment include, but are not limited to, Rhegmatogenous retinal detachment (RRD), tractional retinal detachment, exudative detachment. As used herein, Rhegmatogenous retinal detachment (RRD) generally includes and refers to a state where the vitreous humor becomes less viscous, pulling away from the retina, and adherence of the vitreous to the retina and detaches the retina during the process of liquification. Additionally, addition trauma to the ocular globe from a blunt force injury can result in Rhegmatogenous retinal detachment (RRD). As used herein, tractional retinal detachment generally includes and refers to the contraction of cellular membranes that can form on the retina as a result of disease process (e.g., neovascularization due to proliferative diabetic retinopathy). As used herein, exudative detachment generally includes and refers to a state where fluid accumulates between the retinal pigment epithelium and the neural retina (e.g., due to inflammation, hemorrhage or tumor growth in the eye). Retinal detachments can be relatively small and discrete or involve large areas of the retina. Retinal detachments can comprise partial or full detachment. In some embodiments, the retinal detachment is chronic. In some embodiments, the retinal detachment is acute. In some instances, the size and location (macular or extra-macular) of the detachment determine the extent of vision loss. In some embodiments, retinal detachment comprises macula-off retinal detachment. In some embodiments, retinal detachment comprises macula-on retinal detachment. In some embodiments, the methods described herein include a method of treating retinal injury, wherein the retinal injury comprises detachment of the neural retina. In certain embodiments, retinal injury includes forces external (e.g., laser light damage, object impact, force trauma, etc.) to an individual that result in injury to the eye (e.g., detachment of the retina). In certain embodiments, retinal injury includes forces internal (e.g., disease, inflammation, aging, etc.) to an individual that result in injury to the eye (e.g., detachment of the retina).

In some embodiments, the methods described herein include a method of treating a loss and/or deterioration in visual acuity. In some embodiments, the methods described herein include a method of inhibiting, reducing, and/or preventing a loss and/or deterioration in in visual acuity. In some embodiments, a loss and/or deterioration in visual acuity is characterized by and/or equivalent to a reduction in visual acuity (e.g., a reduction in visual acuity from 20/100 to 20/200, or a reduction in visual acuity from 20/40 to 20/50, a or a reduction in visual acuity from measured in an increased in logMAR, etc.).

In some embodiments, the methods described herein include a method of treating a loss and/or deterioration in visual function. In some embodiments, the methods described herein include a method of inhibiting, reducing, and/or preventing a loss and/or deterioration in visual function. In some embodiments, a loss in visual function is characterized by and/or equivalent to a reduction in visual function assessments (e.g., visual acuity, field of vision, contrast sensitivity, binocular function, low luminance acuity, low contrast acuity, color vision, perimetry, threshold sensitivity, and/or light/dark adaptation).

In some embodiments, the methods described herein include a method of treating inflammation (e.g., Fas-mediated inflammation) in an eye. In some embodiments, the methods described herein include a method of inhibiting, reducing, and/or preventing inflammation (e.g., Fas-mediated inflammation) in an eye. In some embodiments, the method includes treating symptoms associated with inflammation in the eye (e.g., increased intraocular pressure, loss of and/or decrease in visual acuity, central vision loss, blurred vision, distorted vision, etc.). In some embodiments, retinal inflammation can be determined by observing the symptoms associated with inflammation in the eye (e.g., loss of and/or decrease in visual acuity, central vision loss, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-lb, IP-10, IL-18, MIP-la, IL-6, GFAP, MIP2, MCP-1, or MIP-lb); a Fas-mediated complement-related molecules (complement component 3 (C₃) or complement component 1 q (Clq)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-la), and CCL4 (MIP-lb)); toll-like receptor 4 (TLR4); interleukin cytokines (e.g., IL-lb, IL-18, and IL-6); TNF superfamily cytokines (e.g., TNFa); or GFAP.

In certain instances, treating inflammation in the eye and/or a detached neural retina is useful for treating for retinal cell loss. In certain instances, inhibiting, reducing, and/or preventing inflammation in the eye is useful for treating, inhibiting, reducing, and/or preventing retinal cell loss. In some embodiments, the methods described herein include a method of treating retinal cell loss (e.g., retinal pigment epithelial cells and/or photoreceptors) in an eye. In some embodiments, the methods described herein include a method of inhibiting, reducing, and/or preventing retinal cell loss (e.g., retinal pigment epithelial cells and/or photoreceptors) in an eye. In some embodiments, the method includes treating symptoms associated with retinal cell loss (e.g., retinal pigment epithelial cells and/or photoreceptors) in the eye (e.g., loss of and/or decrease in visual acuity, central vision loss, blurred vision, distorted vision, etc.). In some embodiments, the method includes inhibiting, reducing, and/or preventing symptoms associated with retinal cell loss (e.g., retinal pigment epithelial cells and/or photoreceptors) in the eye (e.g., loss of and/or decrease in visual acuity, central vision loss, blurred vision, distorted vision, etc.).

In certain instances, treating inflammation in the eye, a detached neural retina, and/or retinal cell loss. In certain instances, inhibiting, reducing, and/or preventing inflammation in the eye and/or retinal cell loss is useful for treating, inhibiting, reducing the pathology of, and/or preventing an ocular disease, disorder, or condition. As used herein, ocular disease, disorder, or condition generally includes and/or refers to a disorder or pathological condition of the eye which is not normal to an eye in a healthy state. In some embodiments, the methods described herein include a method of treating an ocular disease, disorder, or condition. In some embodiments, the methods described herein include a method of inhibiting, reducing, and/or preventing an ocular disease, disorder, or condition. In some embodiments, the method includes treating symptoms associated with an ocular disease, disorder, or condition (e.g., loss of and/or decrease in visual acuity, central vision loss, blurred vision, distorted vision, etc.). In some embodiments, the method includes inhibiting, reducing, and/or preventing symptoms associated with an ocular disease, disorder, or condition (e.g., loss of and/or decrease in visual acuity, central vision loss, blurred vision, distorted vision, etc.). In some embodiments, the ocular disease, disorder, or condition comprises the loss and/or death of photoreceptors.

In some embodiments, the methods described herein comprise administering a Fas-inhibitor (e.g., a Fas-inhibiting peptide). In some embodiments, the methods described herein comprise administering a Fas-inhibiting peptide (e.g., a peptide comprising the amino acid sequence HHIYLGAVNYIY) or a pharmaceutically-acceptable salt thereof. In some embodiments, the methods described herein comprise administering the peptide (e.g., a composition comprising the peptide) comprising the amino acid sequence HHIYLGAVNYIY or variant sequence thereof, or a pharmaceutically-acceptable salt of the peptide. In some embodiments, the methods described herein comprise administering the peptide (e.g., a composition comprising the peptide) having the structure of any one of Formulas I-IX or a pharmaceutically-acceptable salt thereof. In some embodiments, the methods described herein comprise administering the peptide (e.g., a composition comprising the peptide) having the structure of Formula I or a pharmaceutically-acceptable salt thereof. In some embodiments, the methods described herein comprise administering the peptide (e.g., a composition comprising the peptide) having the structure of Formula III or a pharmaceutically-acceptable salt thereof.

Provided herein are methods comprising: administering a Fas inhibitor to an eye of an individual and performing surgery (e.g., standard of care surgery) on the eye of the individual. Also provided are methods comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method comprising: administering the peptide (e.g., a composition comprising the peptide) or the pharmaceutically-acceptable salt thereof to an eye of an individual and performing surgery (e.g., standard of care surgery) on the eye of the individual.

In some embodiments, the method is a method of treating a retinal detachment. In some embodiments, the method is a method of treating retinal detachment. In certain embodiments, retinal detachment is rhegmatogenous retinal detachment (RRD). In certain embodiments, retinal detachment is macula-off rhegmatogenous retinal detachment (RRD). In certain embodiments, retinal detachment is macula-on rhegmatogenous retinal detachment (RRD). In certain embodiments, retinal detachment is tractional retinal detachment. In certain embodiments, retinal detachment is exudative detachment. In certain embodiments, retinal detachment is a result of retinal injury. In some embodiments, the method is a method of treating a loss and/or deterioration in visual acuity. In some embodiments, the method is a method of inhibiting, reducing, and/or preventing a loss and/or deterioration in in visual acuity. In some embodiments, the method is method of treating a loss and/or deterioration in visual function. In some embodiments, the method is a method of inhibiting, reducing, and/or preventing a loss and/or deterioration in visual function. In some embodiments, the method is a method of treating inflammation (e.g., Fas-mediated inflammation) in an eye. In some embodiments, the method is a method of inhibiting, reducing, and/or preventing inflammation (e.g., Fas-mediated inflammation) in an eye. In some embodiments, the method is a method of treating photoreceptor cell loss in an eye. In some embodiments, the method is a method of inhibiting, reducing, and/or preventing photoreceptor cell loss in an eye. In some embodiments, the method is a method of treating an ocular disease, disorder, or condition. In some embodiments, the method is a method of inhibiting progression of an ocular disease, disorder, or condition, or symptoms associated therewith.

Provided herein are methods of treating a retinal detachment, comprising: administering a Fas inhibitor to an eye of an individual and performing surgery (e.g., standard of care surgery) on the eye of the individual. Also provided are methods of treating a retinal detachment, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of treating a retinal detachment, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of treating a retinal detachment, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of treating a retinal detachment, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of treating a retinal detachment, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of treating a retinal detachment, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of treating a retinal detachment, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of treating a retinal detachment, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of treating a retinal detachment, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of treating a retinal detachment, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of treating a retinal detachment, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of treating a retinal detachment, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of treating a retinal detachment, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of treating a retinal detachment, comprising: administering the peptide (e.g., a composition comprising the peptide) or the pharmaceutically-acceptable salt thereof to an eye of an individual and performing surgery (e.g., standard of care surgery) on the eye of the individual.

In certain embodiments, retinal detachment is rhegmatogenous retinal detachment (RRD). In certain embodiments, retinal detachment is macula-off rhegmatogenous retinal detachment (RRD). In certain embodiments, retinal detachment is macula-on rhegmatogenous retinal detachment (RRD). In certain embodiments, retinal detachment is tractional retinal detachment. In certain embodiments, retinal detachment is exudative detachment. In certain embodiments, retinal detachment is a result of retinal injury.

Provided herein are methods of treating a loss and/or deterioration in visual acuity, comprising: administering a Fas inhibitor to an eye of an individual and performing surgery (e.g., standard of care surgery) on the eye of the individual. Also provided are methods of treating a loss and/or deterioration in visual acuity, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of treating a loss and/or deterioration in visual acuity, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of treating a loss and/or deterioration in visual acuity, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of treating a loss and/or deterioration in visual acuity, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of treating a loss and/or deterioration in visual acuity, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of treating a loss and/or deterioration in visual acuity, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of treating a loss and/or deterioration in visual acuity, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of treating a loss and/or deterioration in visual acuity, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of treating a loss and/or deterioration in visual acuity, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of treating a loss and/or deterioration in visual acuity, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of treating a loss and/or deterioration in visual acuity, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of treating a loss and/or deterioration in visual acuity, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of treating a loss and/or deterioration in visual acuity, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of treating a loss and/or deterioration in visual acuity, comprising: administering the peptide (e.g., a composition comprising the peptide) or the pharmaceutically-acceptable salt thereof to an eye of an individual and performing surgery (e.g., standard of care surgery) on the eye of the individual.

Provided herein are methods of inhibiting, reducing, and/or preventing a loss and/or deterioration in in visual acuity, comprising: administering a Fas inhibitor to an eye of an individual and performing surgery (e.g., standard of care surgery) on the eye of the individual. Also provided are methods of inhibiting, reducing, and/or preventing a loss and/or deterioration in in visual acuity, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of inhibiting, reducing, and/or preventing a loss and/or deterioration in in visual acuity, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of inhibiting, reducing, and/or preventing a loss and/or deterioration in in visual acuity, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are a method of inhibiting, reducing, and/or preventing a loss and/or deterioration in in visual acuity, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of inhibiting, reducing, and/or preventing a loss and/or deterioration in in visual acuity, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of inhibiting, reducing, and/or preventing a loss and/or deterioration in in visual acuity, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of inhibiting, reducing, and/or preventing a loss and/or deterioration in in visual acuity, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of inhibiting, reducing, and/or preventing a loss and/or deterioration in in visual acuity, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of inhibiting, reducing, and/or preventing a loss and/or deterioration in in visual acuity, comprising: administering the peptide (e.g., a composition comprising the peptide) or the pharmaceutically-acceptable salt thereof to an eye of an individual and performing surgery (e.g., standard of care surgery) on the eye of the individual.

Provided herein are methods of inhibiting, reducing, and/or preventing a loss and/or deterioration in visual function, comprising: administering a Fas inhibitor to an eye of an individual and performing surgery (e.g., standard of care surgery) on the eye of the individual. Also provided are methods of inhibiting, reducing, and/or preventing a loss and/or deterioration in visual function, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of inhibiting, reducing, and/or preventing a loss and/or deterioration in visual function, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of inhibiting, reducing, and/or preventing a loss and/or deterioration in visual function, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of inhibiting, reducing, and/or preventing a loss and/or deterioration in visual function, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of inhibiting, reducing, and/or preventing a loss and/or deterioration in visual function, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of inhibiting, reducing, and/or preventing a loss and/or deterioration in visual function, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of inhibiting, reducing, and/or preventing a loss and/or deterioration in visual function, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of inhibiting, reducing, and/or preventing a loss and/or deterioration in visual function, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of inhibiting, reducing, and/or preventing a loss and/or deterioration in visual function, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of inhibiting, reducing, and/or preventing a loss and/or deterioration in visual function, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of inhibiting, reducing, and/or preventing a loss and/or deterioration in visual function, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of inhibiting, reducing, and/or preventing a loss and/or deterioration in visual function, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of inhibiting, reducing, and/or preventing a loss and/or deterioration in visual function, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of inhibiting, reducing, and/or preventing a loss and/or deterioration in visual function, comprising: administering the peptide (e.g., a composition comprising the peptide) or the pharmaceutically-acceptable salt thereof to an eye of an individual and performing surgery (e.g., standard of care surgery) on the eye of the individual.

Provided herein are methods of inhibiting, reducing, and/or preventing photoreceptor cell loss, comprising: administering a Fas inhibitor to an eye of an individual and performing surgery (e.g., standard of care surgery) on the eye of the individual. Also provided are methods of inhibiting, reducing, and/or preventing photoreceptor cell loss, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of inhibiting, reducing, and/or preventing photoreceptor cell loss, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of inhibiting, reducing, and/or preventing photoreceptor cell loss, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of inhibiting, reducing, and/or preventing photoreceptor cell loss, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of inhibiting, reducing, and/or preventing photoreceptor cell loss, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of inhibiting, reducing, and/or preventing photoreceptor cell loss, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of inhibiting, reducing, and/or preventing photoreceptor cell loss, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of inhibiting, reducing, and/or preventing photoreceptor cell loss, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of inhibiting, reducing, and/or preventing photoreceptor cell loss, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of inhibiting, reducing, and/or preventing photoreceptor cell loss, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of inhibiting, reducing, and/or preventing photoreceptor cell loss, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of inhibiting, reducing, and/or preventing photoreceptor cell loss, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of inhibiting, reducing, and/or preventing photoreceptor cell loss, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of inhibiting, reducing, and/or preventing photoreceptor cell loss, comprising: administering the peptide (e.g., a composition comprising the peptide) or the pharmaceutically-acceptable salt thereof to an eye of an individual and performing surgery (e.g., standard of care surgery) on the eye of the individual.

Provided herein are methods of treating an ocular disease, comprising: administering a Fas inhibitor to an eye of an individual and performing surgery (e.g., standard of care surgery) on the eye of the individual. Also provided are methods of treating an ocular disease, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of treating an ocular disease, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of treating an ocular disease, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of treating an ocular disease, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of treating an ocular disease, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of treating an ocular disease, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of treating an ocular disease, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of treating an ocular disease, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of treating an ocular disease, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of treating an ocular disease, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of treating an ocular disease, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of treating an ocular disease, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of treating an ocular disease, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of treating an ocular disease, comprising: administering the peptide (e.g., a composition comprising the peptide) or the pharmaceutically-acceptable salt thereof to an eye of an individual and performing surgery (e.g., standard of care surgery) on the eye of the individual.

Provided herein are methods of inhibiting progression of an ocular disease, disorder, or condition, or symptoms associated therewith, comprising: administering a Fas inhibitor to an eye of an individual and performing surgery (e.g., standard of care surgery) on the eye of the individual. Also provided are methods of inhibiting progression of an ocular disease, disorder, or condition, or symptoms associated therewith, comprising: administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual. Further provided are methods of inhibiting progression of an ocular disease, disorder, or condition, or symptoms associated therewith, comprising: administering a peptide (e.g., a composition comprising the peptide) comprising the structure of any one of Formulas I-IX or a pharmaceutically-acceptable salt thereof, to an eye of an individual, and performing surgery (e.g., standard of care surgery) on the eye of the individual. Provided are methods of inhibiting progression of an ocular disease, disorder, or condition, or symptoms associated therewith, comprising: administering a peptide (e.g., a composition comprising the peptide) comprising the structure of Formula I or a pharmaceutically-acceptable salt thereof, to an eye of an individual, and performing surgery (e.g., standard of care surgery) on the eye of the individual. Also provided are methods of inhibiting progression of an ocular disease, disorder, or condition, or symptoms associated therewith, comprising: administering a peptide (e.g., a composition comprising the peptide) comprising the structure of Formula III or a pharmaceutically-acceptable salt thereof, to an eye of an individual, and performing surgery (e.g., standard of care surgery) on the eye of the individual.

Provided herein are uses of a Fas inhibitor in a method of inhibiting progression of an ocular disease, disorder, or condition, or symptoms associated therewith, comprising: administering the Fas inhibitor to an eye of an individual and performing surgery (e.g., standard of care surgery) on the eye of the individual. Also provided are uses of a peptide comprising an amino acid sequence HHIYLGAVNYIY or a variant sequence thereof, or a pharmaceutically-acceptable salt of the peptide in a method of inhibiting progression of an ocular disease, disorder, or condition, or symptoms associated therewith, comprising: administering the peptide (e.g., a composition comprising the peptide) or the pharmaceutically-acceptable salt of the peptide to an eye of an individual and performing surgery (e.g., standard of care surgery) on the eye of the individual. Further provided are uses of a peptide having the structure of any one of Formulas I-IX or a pharmaceutically-acceptable salt thereof, in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual, and performing surgery (e.g., standard of care surgery) on the eye of the individual. Provided are uses of a peptide having the structure of Formula I or a pharmaceutically-acceptable salt thereof in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual and performing surgery (e.g., standard of care surgery) on the eye of the individual. Provided are uses of a peptide having the structure of Formula III or a pharmaceutically-acceptable salt thereof in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual and performing surgery (e.g., standard of care surgery) on the eye of the individual.

Provided herein are Fas inhibitors for use in a method of inhibiting progression of an ocular disease, disorder, or condition, or symptoms associated therewith, comprising: administering the Fas inhibitor to an eye of an individual and performing surgery (e.g., standard of care surgery) on the eye of the individual. Also provided is a peptide comprising an amino acid sequence HHIYLGAVNYIY or a variant sequence thereof, or a pharmaceutically-acceptable salt of the peptide for use in a method of inhibiting progression of an ocular disease, disorder, or condition, or symptoms associated therewith, comprising: administering the peptide (e.g., a composition comprising the peptide) or the pharmaceutically-acceptable salt of the peptide to an eye of an individual and performing surgery (e.g., standard of care surgery) on the eye of the individual. Further provided is a peptide having the structure of any one of Formulas I-IX or a pharmaceutically-acceptable salt thereof, for use in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual, and performing surgery (e.g., standard of care surgery) on the eye of the individual. Provided is a peptide having the structure of Formula I or a pharmaceutically-acceptable salt thereof for use in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual and performing surgery (e.g., standard of care surgery) on the eye of the individual. Provided is a peptide having the structure of Formula III or a pharmaceutically-acceptable salt thereof for use in a method of inhibiting progression of an ocular disease, disorder, or condition, or symptoms associated therewith, comprising: administering the peptide (e.g., a composition comprising the peptide) or the pharmaceutically-acceptable salt thereof to an eye of an individual and performing surgery (e.g., standard of care surgery) on the eye of the individual.

In some embodiments, the surgery (e.g., standard of care surgery) comprises surgically re-attaching the neural retina. Different surgical procedures are generally available for repairing detachment of the neural retina. In certain instances, the surgical procedure depends upon the location and size of the retinal tear and subsequent detachment. For example, in the case of an isolated retinal tear without any associated subretinal fluid (e.g., a tear that has not yet progressed to a detachment), laser photocoagulation is typically used (e.g., a small, specifically designed laser is directed through a lens or ophthalmoscope to cauterize tissue around the tear thus creating scarring that, in essence, ‘welds’ the retina to the retinal pigment epithelium). In addition to laser photocoagulation, in certain instances, cryopexy is used to freeze the retina around the tear also creating scarring around the tear that holds the retina in place. In instances where the tearing has progressed to a detachment, other techniques are indicated. In certain instances, pneumatic retinopexy is performed wherein a small amount of inert gas (SF6 or C2F8) is injected into the vitreous as a way to tampon the retinal break and allow for reabsorption of the subretinal fluid by the RPE. In certain instances, a scleral buckle is used alone or in conjunction with surgery, which involves the placement of a silicone band around the eye and under the extraocular rectus muscles. In some instances, the surgery comprises a pars plana vitrectomy (PPV), which involves the removal of the vitreous humor and the use of a gas (C3F8 or SF6) or silicone oil tamponade to provide homogenous pressure upon the retina against the RPE. In such instances, PPV surgery is often combined with scleral buckles to enhance the outcome of intervention. In certain instances, PPV utilizes three mechanisms of treatment (1) removal of a potential causative agent (liquified vitreous), (2) pressing the globe against the retina, and (3) pressing the retina back against the RPE, thereby allowing for re-attachment. In certain instances, any of the surgical procedures described herein can be used in the methods described herein.

In some embodiments, the surgery comprises a vitrectomy, a scleral buckle, a pneumatic retinopexy, a laser surgery, or a combination thereof. In some embodiments, the surgery comprises a vitrectomy. In certain embodiments, the vitrectomy is a partial vitrectomy. In certain embodiments, the vitrectomy is a full vitrectomy. In certain embodiments, the vitrectomy comprises the use of silicone oil (e.g., the vitreous is replaced by silicone oil). In certain embodiments, the vitrectomy comprises the use of gas (e.g., the vitreous is replaced by a gas). In some embodiments, the surgery comprises a scleral buckle. In some embodiments, the surgery comprises a pneumatic retinopexy. In some embodiments, the surgery comprises a laser surgery. In some embodiments, the surgery comprises or a combination of one or more of the following: a vitrectomy, a scleral buckle, a pneumatic retinopexy, and a laser surgery.

In some embodiments, the individual has a detached neural retina. In certain instances, the extent of detachment is associated with a greater deterioration in visual acuity and visual function after surgery alone. In such instances, the efficacy of treating or inhibiting deterioration in visual acuity with surgery generally decreases with the size or extent of detachment, however, methods provided herein are useful in inhibiting deterioration in visual acuity and visual function in patients having larger detachments. In certain embodiments, the neural retina is fully detached. In certain embodiments, the neural retina is partially detached. In certain embodiments, the neural retina is fully detached. Retinal detachment is generally indicated by clock hours, wherein an extent or size of detachment ranges from one (1) to twelve (12), with twelve being a full detachment. In some embodiments, the extent of detachment is 12. In some embodiments, the extent of detachment is 11 or greater. In some embodiments, the extent of detachment is 10 or greater. In some embodiments, the extent of detachment is 9 or greater. In some embodiments, the extent of detachment is 8 or greater. In some embodiments, the extent of detachment is 7 or greater. In some embodiments, the extent of detachment is 6 or greater. In some embodiments, the extent of detachment is 5 or greater. In some embodiments, the extent of detachment is 4 or greater. In some embodiments, the extent of detachment is 3 or greater. In some embodiments, the extent of detachment is 2 or greater. In some embodiments, the extent of detachment is 1 or greater.

In some embodiments, the extent of detachment is about 1 to about 12. In some embodiments, the extent of detachment is about 1 to about 2, about 1 to about 3, about 1 to about 4, about 1 to about 5, about 1 to about 6, about 1 to about 7, about 1 to about 8, about 1 to about 9, about 1 to about 10, about 1 to about 11, about 1 to about 12, about 2 to about 3, about 2 to about 4, about 2 to about 5, about 2 to about 6, about 2 to about 7, about 2 to about 8, about 2 to about 9, about 2 to about 10, about 2 to about 11, about 2 to about 12, about 3 to about 4, about 3 to about 5, about 3 to about 6, about 3 to about 7, about 3 to about 8, about 3 to about 9, about 3 to about 10, about 3 to about 11, about 3 to about 12, about 4 to about 5, about 4 to about 6, about 4 to about 7, about 4 to about 8, about 4 to about 9, about 4 to about 10, about 4 to about 11, about 4 to about 12, about 5 to about 6, about 5 to about 7, about 5 to about 8, about 5 to about 9, about 5 to about 10, about 5 to about 11, about 5 to about 12, about 6 to about 7, about 6 to about 8, about 6 to about 9, about 6 to about 10, about 6 to about 11, about 6 to about 12, about 7 to about 8, about 7 to about 9, about 7 to about 10, about 7 to about 11, about 7 to about 12, about 8 to about 9, about 8 to about 10, about 8 to about 11, about 8 to about 12, about 9 to about 10, about 9 to about 11, about 9 to about 12, about 10 to about 11, about 10 to about 12, or about 11 to about 12. In some embodiments, the extent of detachment is about 1, about 2, about 3, about 4, about 5, about 6, about 7, about 8, about 9, about 10, about 11, or about 12. In some embodiments, the extent of detachment is at least about 1, about 2, about 3, about 4, about 5, about 6, about 7, about 8, about 9, about 10, or about 11.

In certain instances, increasing heights of detachment is associated with a greater deterioration in visual acuity and visual function after surgery alone. In such instances, the efficacy of treating or inhibiting deterioration in visual acuity with surgery generally decreases with an increase in the height of detachment, however, methods provided herein are useful in inhibiting deterioration in visual acuity and visual function in patients having larger detachment heights. In some embodiments, the height of detachment is between 0 and 1 millimeter(s). In some embodiments, the height of detachment is 1 millimeter(s) or greater. In some embodiments, the height of detachment is 2 millimeter(s) or greater. In some embodiments, the height of detachment is 3 millimeter(s) or greater. In some embodiments, the height of detachment is 4 millimeter(s) or greater. In some embodiments, the height of detachment is 5 millimeter(s) or greater. In some embodiments, the height of detachment is 7 millimeter(s) or greater.

In some embodiments, surgery is performed about (z) day(s) after administering the composition comprising the peptide, wherein (z) is at least one. In certain instances, providing a Fas inhibitor is prior to surgery provides improved outcomes (e.g., visual acuity and visual function) compared to surgery alone. In certain instances, the time of detachment is associated with a greater deterioration in visual acuity and visual function after surgery alone. Furthermore, the efficacy of treating or inhibiting deterioration in visual acuity with surgery generally decreases with the time of detachment, however, patients are not always to undergo surgery immediately or within a reasonable time (e.g., less than about 8 days). In such instances, the methods provided herein are useful in safely extending the time in which surgery is performed while still inhibiting deterioration in visual acuity and visual function in patients. In some embodiments, (z) is 1 or greater (e.g., about 1 day or greater). In some embodiments, (z) is 2 or greater (e.g., about 2 days or greater). In some embodiments, (z) is 3 or greater (e.g., about 3 days or greater). In some embodiments, (z) is 4 or greater (e.g., about 4 days or greater). In some embodiments, (z) is 5 or greater (e.g., about 5 days or greater). In some embodiments, (z) is 6 or greater (e.g., about 6 days or greater). In some embodiments, (z) is 7 or greater (e.g., about 7 days or greater). In some embodiments, (z) is 8 or greater (e.g., about 8 days or greater). In some embodiments, (z) is 10 or greater (e.g., about 10 days or greater). In some embodiments, (z) is 14 or greater (e.g., about 14 days or greater).

In certain embodiments, (z) is about 0.5 to about 21. In certain embodiments, (z) is about 0.5 to about 1, about 0.5 to about 2, about 0.5 to about 3, about 0.5 to about 4, about 0.5 to about 5, about 0.5 to about 7, about 0.5 to about 10, about 0.5 to about 14, about 0.5 to about 21, about 1 to about 2, about 1 to about 3, about 1 to about 4, about 1 to about 5, about 1 to about 7, about 1 to about 10, about 1 to about 14, about 1 to about 21, about 2 to about 3, about 2 to about 4, about 2 to about 5, about 2 to about 7, about 2 to about 10, about 2 to about 14, about 2 to about 21, about 3 to about 4, about 3 to about 5, about 3 to about 7, about 3 to about 10, about 3 to about 14, about 3 to about 21, about 4 to about 5, about 4 to about 7, about 4 to about 10, about 4 to about 14, about 4 to about 21, about 5 to about 7, about 5 to about 10, about 5 to about 14, about 5 to about 21, about 7 to about 10, about 7 to about 14, about 7 to about 21, about 10 to about 14, about 10 to about 21, or about 14 to about 21. In certain embodiments, (z) is about 0.5, about 1, about 2, about 3, about 4, about 5, about 7, about 10, about 14, or about 21. In certain embodiments, (z) is at least about 0.5, about 1, about 2, about 3, about 4, about 5, about 7, about 10, or about 14.

In some embodiments, surgery is performed about (h) hour(s) after administering the composition comprising the peptide, wherein (h) is at least one. In some embodiments, (h) is 1 or greater (e.g., about 1 hour or greater). In some embodiments, (h) is 2 or greater (e.g., about 2 hours or greater). In some embodiments, (h) is 3 or greater (e.g., about 3 hours or greater). In some embodiments, (h) is 6 or greater (e.g., about 6 hours or greater). In some embodiments, (h) is 12 or greater (e.g., about 12 hours or greater). In some embodiments, (h) is 18 or greater (e.g., about 18 hours or greater). In some embodiments, (h) is 24 or greater (e.g., about 24 hours or greater). In some embodiments, (h) is 30 or greater (e.g., about 30 hours or greater). In some embodiments, (h) is 36 or greater (e.g., about 36 hours or greater). In some embodiments, (h) is 48 or greater (e.g., about 48 hours or greater).

In certain embodiments, (h) is about 1 to about 42. In certain embodiments, (h) is about 1 to about 2, about 1 to about 3, about 1 to about 6, about 1 to about 12, about 1 to about 18, about 1 to about 24, about 1 to about 30, about 1 to about 36, about 1 to about 42, about 2 to about 3, about 2 to about 6, about 2 to about 12, about 2 to about 18, about 2 to about 24, about 2 to about 30, about 2 to about 36, about 2 to about 42, about 3 to about 6, about 3 to about 12, about 3 to about 18, about 3 to about 24, about 3 to about 30, about 3 to about 36, about 3 to about 42, about 6 to about 12, about 6 to about 18, about 6 to about 24, about 6 to about 30, about 6 to about 36, about 6 to about 42, about 12 to about 18, about 12 to about 24, about 12 to about 30, about 12 to about 36, about 12 to about 42, about 18 to about 24, about 18 to about 30, about 18 to about 36, about 18 to about 42, about 24 to about 30, about 24 to about 36, about 24 to about 42, about 30 to about 36, about 30 to about 42, or about 36 to about 42. In certain embodiments, (h) is about 1, about 2, about 3, about 6, about 12, about 18, about 24, about 30, about 36, or about 42. In certain embodiments, (h) is at least about 1, about 2, about 3, about 6, about 12, about 18, about 24, about 30, or about 36.

As used herein, the term “best corrected visual acuity” or “BCVA”, generally refers to the minimum angle of resolution subtended by a certain number of arc minutes. In certain instances, the subtended visual angle of an object is the angle formed by rays projecting from the eye to the top and bottom (or left and right sides) of an object. In such instances, such visual angles are used to indicate the size of the retinal image of the object (e.g., the larger the visual angle, the larger the retinal image size is). In certain instances, the visual angle is influenced by two parameters: the size of the object and the distance of the object from the eye. Bigger objects cast larger images on the retina than smaller objects. Thus, the larger the object is, the larger its visual angle will be. Closer objects cast larger images on the retina than smaller objects. Thus, the closer the object is to the eye, the larger its visual angle will be. By way of example, standard vision is thus the ability to distinguish features separated by 1 minute of arc. By way of further example, the ability to distinguish a set of bars separated by 1 arc minute is 20/20 vision, 2 arc minutes is 20/40 vision. In some embodiments, visual acuity is measured by a Snellen chart, ETDRS chart, Landolt C chart, Tumbling E chart, HOTV chart, and/or logMAR (log minimum angle of resolution). FIG. 2A-2B show an exemplary, but non-limiting, conversion chart for comparing BCVA values.

In certain instances, the extent of loss of visual acuity due to the retinal detachment is associated with ultimate visual outcomes after treatment with surgery (e.g., a worsened baseline visual acuity is associated with decreased visual acuity). In certain instances, the methods described herein are useful for treating patient's having extensive baseline vision loss (e.g., acuity limited to hand motion or counting fingers). In some embodiments, the individual has baseline (e.g., prior to treatment) best corrected visual acuity of detecting hand motion. In some embodiments, the individual has baseline (e.g., prior to treatment) best corrected visual acuity of counting fingers. In some embodiments, the individual has baseline (e.g., prior to treatment) best corrected visual acuity of 20/100 or worse (e.g., a worse visual acuity such as 20/200) on a Snellen chart. In some embodiments, the individual has baseline (e.g., prior to treatment) best corrected visual acuity of logMAR 3 or greater (e.g., a worsed visual acuity such as logMAR 3.5). In some embodiments, the individual has baseline (e.g., prior to treatment) best corrected visual acuity of logMAR 2 or greater. In some embodiments, the individual has baseline (e.g., prior to treatment) best corrected visual acuity of logMAR 1 or greater. In some embodiments, the individual has baseline (e.g., prior to treatment) best corrected visual acuity of logMAR 0.5 or greater.

In some embodiments, the individual has baseline (e.g., prior to treatment) BCVA of 20/200 or worse (e.g., a worse visual acuity such as 20/400). In some embodiments, the individual has baseline (e.g., prior to treatment) BCVA of 20/100 or worse. In some embodiments, the individual has baseline (e.g., prior to treatment) BCVA of 20/70 or worse. In some embodiments, the individual has baseline (e.g., prior to treatment) BCVA of 20/50 or worse.

In some embodiments, the individual has baseline (e.g., prior to treatment) visual angle in minutes of 32 or greater (e.g., a worse visual acuity such as a visual angle in minutes of 40). In some embodiments, the individual has baseline (e.g., prior to treatment) BCVA in visual angle in minutes of 24 or greater. In some embodiments, the individual has baseline (e.g., prior to treatment) BCVA in visual angle in minutes of 16 or greater. In some embodiments, the individual has baseline (e.g., prior to treatment) BCVA in visual angle in minutes of 8 or greater. In some embodiments, the individual has baseline (e.g., prior to treatment) BCVA in visual angle in minutes of 4 or greater. In some embodiments, the individual has baseline (e.g., prior to treatment) BCVA in visual angle in minutes of 2 or greater.

In certain embodiments, the individual has baseline (e.g., prior to treatment) BCVA in visual angle in minutes of about 1 to about 32. In certain embodiments, the individual has baseline (e.g., prior to treatment) BCVA in visual angle in minutes of about 1 to about 2, about 1 to about 4, about 1 to about 8, about 1 to about 12, about 1 to about 16, about 1 to about 20, about 1 to about 24, about 1 to about 28, about ito about 32, about 2 to about 4, about 2 to about 8, about 2 to about 12, about 2 to about 16, about 2 to about 20, about 2 to about 24, about 2 to about 28, about 2 to about 32, about 4 to about 8, about 4 to about 12, about 4 to about 16, about 4 to about 20, about 4 to about 24, about 4 to about 28, about 4 to about 32, about 8 to about 12, about 8 to about 16, about 8 to about 20, about 8 to about 24, about 8 to about 28, about 8 to about 32, about 12 to about 16, about 12 to about 20, about 12 to about 24, about 12 to about 28, about 12 to about 32, about 16 to about 20, about 16 to about 24, about 16 to about 28, about 16 to about 32, about 20 to about 24, about 20 to about 28, about 20 to about 32, about 24 to about 28, about 24 to about 32, or about 28 to about 32. In certain embodiments, the individual has baseline (e.g., prior to treatment) BCVA in visual angle in minutes of about 1, about 2, about 4, about 8, about 12, about 16, about 20, about 24, about 28, or about 32. In certain embodiments, the individual has baseline (e.g., prior to treatment) BCVA in visual angle in minutes of at least about 1, about 2, about 4, about 8, about 12, about 16, about 20, about 24, or about 28. In certain embodiments, the individual has baseline (e.g., prior to treatment) BCVA in visual angle in minutes of at most about 2, about 4, about 8, about 12, about 16, about 20, about 24, about 28, or about 32.

In some embodiments, the subject has central vision loss. In certain instances, providing a Fas inhibitor is prior to surgery provides improved outcomes (e.g., visual acuity and visual function) in patients having central vision loss when compared to surgery alone. In such certain instances, providing a Fas inhibitor is prior to surgery provides improved outcomes (e.g., visual acuity and visual function) in patients having prolonged central vision loss (e.g., 3 days, 5 days, 7 days, 10 days, 14 days, 21 days, etc.) when compared to surgery alone. In certain instances, the time of detachment is measured by the of central vision loss. In certain instances, the time of central vision loss is associated with a greater deterioration in visual acuity and visual function after surgery alone. Furthermore, the efficacy of treating or inhibiting deterioration in visual acuity with surgery generally decreases as the time of central vision loss increases. In such instances, the methods provided herein are useful in treating patients having prolonged central vision loss (e.g., 3 days, 5 days, 7 days, 10 days, 14 days, 21 days, etc.). In some embodiments, the eye of the individual has a retina that is detached, and wherein the retina has been detached for about (y) or more days (e.g., prior to administering the composition comprising the Fas inhibitor or performing surgery), wherein (y) is at least one. In some embodiments, the eye of the individual has central vision loss, and wherein the central vision loss has been present for about (y) or more days (e.g., prior to administering the composition comprising the Fas inhibitor or performing surgery), wherein (y) is at least one. In certain embodiments, the eye of the individual has a retinal injury or a symptom thereof, and wherein the retinal injury or the symptom thereof has been present for about (y) or more days (e.g., prior to administering the composition comprising the Fas inhibitor or performing surgery), wherein (y) is at least one.

In certain embodiments, (y) is 1 (e.g., about 1 or more days). In certain embodiments, (y) is 3 (e.g., about 3 or more days). In certain embodiments, (y) is 5 (e.g., about 5 or more days). In certain embodiments, (y) is 7 (e.g., about 7 or more days). In certain embodiments, (y) is 10 (e.g., about 10 or more days). In certain embodiments, (y) is 14 (e.g., about 14 or more days). In certain embodiments, (y) is 20 (e.g., about 20 or more days). In certain embodiments, (y) is 30 (e.g., about 30 or more days). In certain embodiments, (y) is about 0.5 days to about 40 days. In certain embodiments, (y) is about 0.5 days to about 1 day, about 0.5 days to about 3 days, about 0.5 days to about 5 days, about 0.5 days to about 7 days, about 0.5 days to about 10 days, about 0.5 days to about 14 days, about 0.5 days to about 20 days, about 0.5 days to about 30 days, about 0.5 days to about 40 days, about 1 day to about 3 days, about 1 day to about 5 days, about 1 day to about 7 days, about 1 day to about 10 days, about 1 day to about 14 days, about 1 day to about 20 days, about 1 day to about 30 days, about 1 day to about 40 days, about 3 days to about 5 days, about 3 days to about 7 days, about 3 days to about 10 days, about 3 days to about 14 days, about 3 days to about 20 days, about 3 days to about 30 days, about 3 days to about 40 days, about 5 days to about 7 days, about 5 days to about 10 days, about 5 days to about 14 days, about 5 days to about 20 days, about 5 days to about 30 days, about 5 days to about 40 days, about 7 days to about 10 days, about 7 days to about 14 days, about 7 days to about 20 days, about 7 days to about 30 days, about 7 days to about 40 days, about 10 days to about 14 days, about 10 days to about 20 days, about 10 days to about 30 days, about 10 days to about 40 days, about 14 days to about 20 days, about 14 days to about 30 days, about 14 days to about 40 days, about 20 days to about 30 days, about 20 days to about 40 days, or about 30 days to about 40 days. In certain embodiments, (y) is about 0.5 days, about 1 day, about 3 days, about 5 days, about 7 days, about 10 days, about 14 days, about 20 days, about 30 days, or about 40 days. In certain embodiments, (y) is at least about 0.5 days, about 1 day, about 3 days, about 5 days, about 7 days, about 10 days, about 14 days, about 20 days, about 30 days, or about 40 days.

In some embodiments, the subject has symptoms associated with a detached neural retina (e.g., a loss or reduction in visual function). In certain instances, the time of detachment is measured by the onset of symptoms associated with a detached or detaching retina. In certain instances, the time of central vision loss is associated with a greater deterioration in visual acuity and visual function after surgery alone. In such instances, the efficacy of treating or inhibiting deterioration in visual acuity with surgery generally decreases as the time of symptoms associated with a detached or detaching retina increases, however, the methods provided herein are useful in treating patients having prolonged symptoms associated with a detached or detaching retina (e.g., 3 days, 5 days, 7 days, 10 days, 14 days, 21 or more days, etc.). In some embodiments, the eye of the individual has symptoms associated with a detached neural retina (e.g., a loss or reduction in visual function), and wherein the symptoms associated with a detached neural retina (e.g., a loss or reduction in visual function) have been present for about (s) or more days (e.g., prior to administering the composition comprising the Fas inhibitor or performing surgery), wherein (s) is at least one.

In certain embodiments, (s) is 1 (e.g., about 1 or more days). In certain embodiments, (s) is 3 (e.g., about 3 or more days). In certain embodiments, (s) is 5 (e.g., about 5 or more days). In certain embodiments, (s) is 7 (e.g., about 7 or more days). In certain embodiments, (s) is 10 (e.g., about 10 or more days). In certain embodiments, (s) is 14 (e.g., about 14 or more days). In certain embodiments, (s) is 20 (e.g., about 20 or more days). In certain embodiments, (s) is 30 (e.g., about 30 or more days).

In certain embodiments, (s) is about 1 day to about 40 days. In certain embodiments, (s) is about 1 day to about 3 days, about 1 day to about 5 days, about 1 day to about 7 days, about 1 day to about 10 days, about 1 day to about 14 days, about 1 day to about 20 days, about 1 day to about 30 days, about 1 day to about 40 days, about 3 days to about 5 days, about 3 days to about 7 days, about 3 days to about 10 days, about 3 days to about 14 days, about 3 days to about 20 days, about 3 days to about 30 days, about 3 days to about 40 days, about 5 days to about 7 days, about 5 days to about 10 days, about 5 days to about 14 days, about 5 days to about 20 days, about 5 days to about 30 days, about 5 days to about 40 days, about 7 days to about 10 days, about 7 days to about 14 days, about 7 days to about 20 days, about 7 days to about 30 days, about 7 days to about 40 days, about 10 days to about 14 days, about 10 days to about 20 days, about 10 days to about 30 days, about 10 days to about 40 days, about 14 days to about 20 days, about 14 days to about 30 days, about 14 days to about 40 days, about 20 days to about 30 days, about 20 days to about 40 days, or about 30 days to about 40 days. In certain embodiments, (s) is about 1 day, about 3 days, about 5 days, about 7 days, about 10 days, about 14 days, about 20 days, about 30 days, or about 40 days. In certain embodiments, (s) is at least about 1 day, about 3 days, about 5 days, about 7 days, about 10 days, about 14 days, about 20 days, about 30 days, or about 40 days.

In certain instances, increased age of the individual can be associated with a greater deterioration in visual acuity and visual function. In such instances, the efficacy of treating or inhibiting deterioration in visual acuity with surgery generally decreases as the age of an individual having a detached or detaching retina increases, however, the methods provided herein are useful in treating patients across age groups. In some embodiments, the individual is 40 years of age or older. In some embodiments, the individual is about 50 years of age or older. In some embodiments, the individual is about 55 years of age or older. In some embodiments, the individual is about 60 years of age or older. In some embodiments, the individual is about 65 years of age or older. In some embodiments, the individual is 70 years of age or older. In some embodiments, the individual is about 75 years of age or older.

In certain embodiments, the individual is about 20 years old to about 80 years old. In certain embodiments, the individual is about 20 years old to about 30 years old, about 20 years old to about 40 years old, about 20 years old to about 50 years old, about 20 years old to about 60 years old, about 20 years old to about 70 years old, about 20 years old to about 80 years old, about 30 years old to about 40 years old, about 30 years old to about 50 years old, about 30 years old to about 60 years old, about 30 years old to about 70 years old, about 30 years old to about 80 years old, about 40 years old to about 50 years old, about 40 years old to about 60 years old, about 40 years old to about 70 years old, about 40 years old to about 80 years old, about 50 years old to about 60 years old, about 50 years old to about 70 years old, about 50 years old to about 80 years old, about 60 years old to about 70 years old, about 60 years old to about 80 years old, or about 70 years old to about 80 years old. In certain embodiments, the individual is about 20 years old, about 30 years old, about 40 years old, about 50 years old, about 60 years old, about 70 years old, or about 80 years old. In certain embodiments, the individual is at least about 20 years old, about 30 years old, about 40 years old, about 50 years old, about 60 years old, or about 70 years old.

In certain instances, detachment of the neural retina is accompanied by an inflamed state within the eye (e.g., within the retinal tissue). In further instances, inflammation results in or is associated with deterioration of cell death within the eye (e.g., photoreceptor cell death). In these instances, surgery alone does not target reducing inflammation within the eye (e.g., within the retinal tissue). In such instances, the methods provided herein are useful in treating and/or inhibiting inflammation in the eye of the individual. In some embodiments, the eye of the individual comprises inflammation (e.g., an increased amount of one or more biomarkers). In certain embodiments, inflammation comprises an increased amount of one or more biomarkers associated with inflammation. In certain embodiments, the increased amount of one or more biomarkers associated with inflammation is relative to an eye not having a detached neural retina or the non-injured eye. In certain embodiments, the one or more biomarkers associated with inflammation (i.e., inflammatory molecules) include, but are not limited to, Fas-mediated inflammation-related molecules (e.g. TNFa, IL-lb, IP-10, IL-18, MIP-la, IL-6, GFAP, MIP2, MCP-1, or MIP-lb); a Fas-mediated complement-related molecules (complement component 3 (C₃) or complement component 1 q (Clq)) Caspase 8; components of the inflammasome (e.g., NLRP3 or NLRP2); C-X-C motif chemokines (e.g., CXCL2 (MIP-2alpha) or CXCL10 (IP-10)); C-X3-C motif chemokines (e.g., CX3CL1 (fractalkine)); C-C motif chemokines (CCL2 (MCP-1), CCL3 (MIP-la), and CCL4 (MIP-1 b)); toll-like receptor 4 (TLR4); interleukin cytokines (e.g., IL-lb, IL-18, and IL-6); TNF superfamily cytokines (e.g., TNFa); or GFAP. In certain embodiments, the presence and/or amount of one or more biomarkers associated with inflammation by a biological assay (e.g., an immunoassay, ELISA-based assay, etc.) detecting the presence of inflammatory molecules (e.g., inflammatory cytokines) in a sample (e.g., vitreous humor sample) taken from the eye.

In some embodiments, the eye of the individual has a cataract. In some embodiments, the eye of the individual potentially has a cataract. In some embodiments, the eye of the individual potentially has a pseudophakic lens. In some embodiments, the individual has a preexisting disease or disorder that includes non-ocular pathologies (e.g., diabetes).

In some embodiments, the composition is administered via intravitreal injection, intracameral injection, injection into the suprachoroidal space, injection in the subtenons space, subconjunctival injection, retrobulbar injection, peribulbar injection, microneedle injection, subretinal injection, or subretinal infusion. In certain embodiments, the composition is administered via intravitreal injection. In certain embodiments, the composition is administered via intracameral injection. In certain embodiments, the composition is administered via injection into the suprachoroidal space. In certain embodiments, the composition is administered via injection in the subtenons space. In certain embodiments, the composition is administered via subconjunctival injection. In certain embodiments, the composition is administered via retrobulbar injection. In certain embodiments, the composition is administered via peribulbar injection. In certain embodiments, the composition is administered via microneedle injection. In certain embodiments, the composition is administered via subretinal injection. In certain embodiments, the composition is administered via subretinal infusion.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, comprises a modified amino acid or a non-natural amino acid. In some embodiments, the peptide comprises an amidated C-terminus. wherein the peptide has the structure of Formula I, or a pharmaceutically-acceptable salt thereof.

In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, a composition comprising the Fas inhibitor (e.g., the Fas inhibiting peptide) is administered to the eye of the individual. In some embodiments, a composition comprising the peptide is administered to the eye of the individual. In some embodiments, the composition further comprises on or more excipients. In some embodiments, the composition further comprises a surfactant. In some embodiments, the surfactant is a non-ionic surfactant.

In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant is a polysorbate. In some embodiments, the surfactant is a polyethoxylated alcohol. In some embodiments, the surfactant is a polyoxyethylene-polyoxypropylene block copolymer. In some embodiments, the surfactant is an oxyethylated tertiary octylphenol formaldehyde polymer.

In some embodiments, the surfactant comprises Polysorbate 20, Poloxamer 407, Tyloxapol, or cremophor. In some embodiments, the surfactant comprises Polysorbate 20. In some embodiments, the surfactant comprises Poloxamer 407. In some embodiments, the surfactant comprises Tyloxapol. In some embodiments, the surfactant comprises cremophor. In some embodiments, the surfactant forms about 0.01% to about 20% weight/weight of the composition. In some embodiments, the surfactant forms about 0.05% to about 10% weight/weight of the composition. In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the composition further comprises a tonicity adjusting agent, a buffering agent, or a combination thereof (e.g., as described herein, including any of the described ranges and/or values within the described ranges). In some embodiments, the composition is buffered at a pH of 2.5 to 7.5.

In some embodiments, the composition comprises 5 micrograms (ug) to 10,000 ug of the peptide. In some embodiments, the composition comprises about 5 ug of the peptide to about 300 ug of the peptide. In some embodiments, the composition comprises at least about 5 ug of the peptide. In some embodiments, the composition comprises at least about 25 ug of the peptide. In some embodiments, the composition comprises at least about 50 ug of the peptide. In some embodiments, the composition comprises at least about 100 ug of the peptide. In some embodiments, the composition comprises at least about 125 ug of the peptide. In some embodiments, the composition comprises at least about 150 ug of the peptide. In some embodiments, the composition comprises at least about 200 ug of the peptide. In some embodiments, the composition comprises at least about 250 ug of the peptide. In some embodiments, the composition comprises at least about 300 ug of the peptide.

In some embodiments, the composition comprises about 5 ug of the peptide to about 10 ug of the peptide, about 5 ug of the peptide to about 25 ug of the peptide, about 5 ug of the peptide to about 50 ug of the peptide, about 5 ug of the peptide to about 75 ug of the peptide, about 5 ug of the peptide to about 100 ug of the peptide, about 5 ug of the peptide to about 150 ug of the peptide, about 5 ug of the peptide to about 200 ug of the peptide, about 5 ug of the peptide to about 250 ug of the peptide, about 5 ug of the peptide to about 300 ug of the peptide, about 10 ug of the peptide to about 25 ug of the peptide, about 10 ug of the peptide to about 50 ug of the peptide, about 10 ug of the peptide to about 75 ug of the peptide, about 10 ug of the peptide to about 100 ug of the peptide, about 10 ug of the peptide to about 150 ug of the peptide, about 10 ug of the peptide to about 200 ug of the peptide, about 10 ug of the peptide to about 250 ug of the peptide, about 10 ug of the peptide to about 300 ug of the peptide, about 25 ug of the peptide to about 50 ug of the peptide, about 25 ug of the peptide to about 75 ug of the peptide, about 25 ug of the peptide to about 100 ug of the peptide, about 25 ug of the peptide to about 150 ug of the peptide, about 25 ug of the peptide to about 200 ug of the peptide, about 25 ug of the peptide to about 250 ug of the peptide, about 25 ug of the peptide to about 300 ug of the peptide, about 50 ug of the peptide to about 75 ug of the peptide, about 50 ug of the peptide to about 100 ug of the peptide, about 50 ug of the peptide to about 150 ug of the peptide, about 50 ug of the peptide to about 200 ug of the peptide, about 50 ug of the peptide to about 250 ug of the peptide, about 50 ug of the peptide to about 300 ug of the peptide, about 75 ug of the peptide to about 100 ug of the peptide, about 75 ug of the peptide to about 150 ug of the peptide, about 75 ug of the peptide to about 200 ug of the peptide, about 75 ug of the peptide to about 250 ug of the peptide, about 75 ug of the peptide to about 300 ug of the peptide, about 100 ug of the peptide to about 150 ug of the peptide, about 100 ug of the peptide to about 200 ug of the peptide, about 100 ug of the peptide to about 250 ug of the peptide, about 100 ug of the peptide to about 300 ug of the peptide, about 150 ug of the peptide to about 200 ug of the peptide, about 150 ug of the peptide to about 250 ug of the peptide, about 150 ug of the peptide to about 300 ug of the peptide, about 200 ug of the peptide to about 250 ug of the peptide, about 200 ug of the peptide to about 300 ug of the peptide, or about 250 ug of the peptide to about 300 ug of the peptide. In some embodiments, the composition comprises about 5 ug of the peptide, about 10 ug of the peptide, about 25 ug of the peptide, about 50 ug of the peptide, about 75 ug of the peptide, about 100 ug of the peptide, about 150 ug of the peptide, about 200 ug of the peptide, about 250 ug of the peptide, or about 300 ug of the peptide.

In some embodiments, the peptide is present at a concentration 0.1 milligrams per milliliter (mg/mL) to 10 mg/mL. In some embodiments, the peptide is present at a concentration 0.1 milligrams per milliliter (mg/mL) to 5.0 mg/mL.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, the term “inhibition” or “inhibiting” includes and/or refers to the reduction or suppression of a given condition, symptom, disorder, or disease, and/or a decrease in the baseline activity of a biological activity or process.

As used herein, the term “treating” or “treatment” of includes and/or refers to ameliorating the disease or disorder or symptoms thereof (e.g., slowing or arresting or reducing the development of the disease or at least one of the clinical symptoms thereof). In certain embodiments, “treating” or “treatment” also includes and/or refers to alleviating or ameliorating at least one physical and/or biological parameters including those which may not be discernible by the patient. In certain embodiments, “treating” or “treatment” includes and/or refers to modulating a disease, disorder, or biological process either physically, (e.g., stabilization of a discernible symptom), physiologically, (e.g., stabilization of a physical and/or biological parameter), or both. In certain embodiments, “treating” or “treatment” includes and/or refers to preventing or delaying the onset or development or progression of the disease or disorder. In certain embodiments, “treating” or “treatment” includes and/or refers to preventing or delaying or inhibiting the deterioration of (i) a healthy physiological state or (ii) a baseline physiological state (e.g., the progression of a disease or disorder).

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, in any instance or embodiment described herein, “comprising” may be replaced with “consisting essentially of” and/or “consisting of”. used herein, in any instance or embodiment described herein, “comprises” may be replaced with “consists essentially of” and/or “consists of”.

### EXAMPLES

### Example I—Improving Visual Acuity in GA Patients

### Objective

The primary objective was to determine safety of a peptide having the structure of Formula III (ONL1204) and select doses in patients having macular degeneration characterized by geographic atrophy (GA). The primary endpoints were the safety profile demonstrated in this study, including: adverse event reporting, clinical evaluations including best corrected visual acuity (BCVA), and clinical evaluation.

### Study Design

Patients showing GA were enrolled in ascending dose groups and received a single intravitreal injection of a peptide having an acetate salt of the structure of Formula III (ONL1204). Baseline best corrected visual acuity (BCVA) was measured prior to treatment with ONL1204. BCVA was then measured 1, 2-, 4-, 12-, and 24-weeks post treatment with ONL1204. The following doses were evaluated: 50 ug and 100 ug provided in vials with concentrations of 0.5 mg/mL and/or 2 mg/mL of Formula III.

### Outcomes

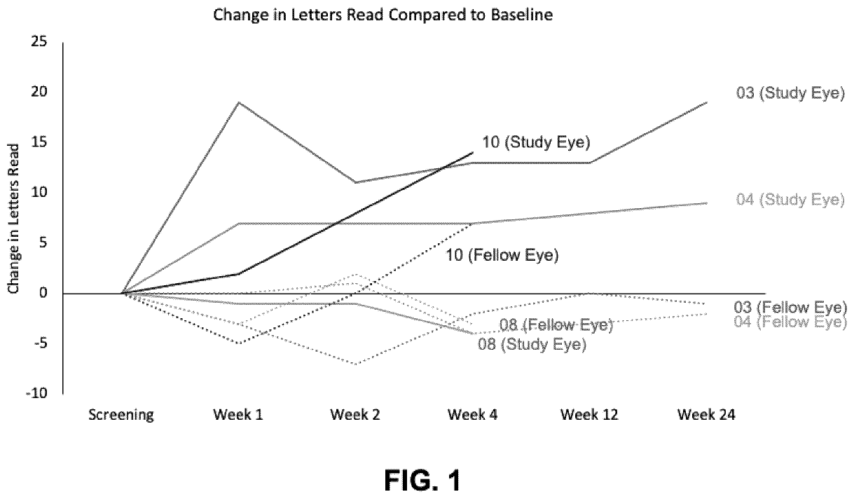

All patients receiving doses comprising 25 ug and 50 ug of ONL1204 showed an improvement in visual outcomes (e.g., visual acuity). FIG. 1 shows study data for the change in best corrected visual acuity (BCVA). An improvement in visual acuity was generally observed for across patients receiving ONL1204, as compared to baseline BCVA (solid lines indicating the study eye and dashed lines indicating the fellow/untreated eye).

### Example 2—Treating Retinal Detachment

### Objective

The primary objective was to determine the Maximum Tolerated Dose (MTD) of a peptide an amino acid sequence HHIYLGAVNYIY and the structure of Formula III (ONL1204) and select doses for further clinical studies. During the study, ocular fluid samples were also collected at the time of study drug injection and at the time of vitrectomy. These samples were used for pharmacokinetic and pharmacodynamic analyses. The primary endpoints were the safety profile demonstrated in this study, including: adverse event reporting, clinical evaluations including best corrected visual acuity (BCVA), and clinical laboratory evaluation. The exploratory endpoints were the pharmacokinetic and pharmacodynamic analyses performed in this study, including: measurement of ONL1204 drug levels in plasma and vitreous fluid for PK analysis, cytokine and other potential molecular biomarker measurement in aqueous and vitreous samples, taken by vitreous tap, and at vitrectomy.

### Study Design

Patients were enrolled in ascending dose groups. After signing informed consent and meeting all eligibility criteria, patients underwent a vitreous tap for cytokine and other potential molecular biomarker analysis followed by a single intravitreal injection of a peptide having an acetate salt of the structure of Formula III (ONL1204). Injection of ONL1204 occurred on the day of diagnosis (Day 1). Safety visits were conducted one day post-injection and as deemed necessary by the examining Investigator (at least 1 visit every 3 days) until Visit 3. At Visit 3 (3+ days post-injection, in accordance with standard of care) patients underwent surgical repair of their retinal detachment with vitrectomy (with or without scleral buckling) and gas or oil tamponade. At the time of surgery, an undiluted vitreous sample was collected for pharmacokinetic and pharmacodynamics analyses, and an aqueous sample was collected for pharmacodynamic analysis. Additional safety visits were targeted for and generally conducted one day post-surgery, 7 days (±3 days) post-surgery, 14 (+4 days) days post-surgery, and Weeks 6, 9, 12 (±7 days each), and Week 24 (+14 days; study exit).

The following doses were evaluated: 25 microgram (ug) and 50 ug provided in vials with concentrations of 0.5 mg/mL and/or 2 mg/mL of Formula III. Therefore, a volume of 0.05 or 0.1 mL was injected into the study eye. Patient participation was approximately 24 weeks (6 months): a single injection treatment and 24 weeks of follow-up.

### Safety

Planned determinations of dose limiting toxicity included consideration of the following events:
1. Ocular inflammation increases by 2 units from pre-injection on the Standardization of Uveitis Nomenclature (SUN) grading scale for aqueous cell or vitreous haze, secondary to inflammation and not the drug formulation itself, identified postinjection of ONL1204 and prior to retinal reattachment surgery;
2. Sustained elevation of IOP characterized as >30 mmHg for 3 consecutive days, postinjection of ONL1204 and prior to retinal reattachment surgery, despite pharmacologic therapy;
3. Reduction in visual acuity from baseline after injection of ONL1204 and prior to retinal reattachment surgery, that in the opinion of the investigator is likely due to the investigational product and results in a: (a) Decrease in visual acuity from 20/200 or 20/400 to light perception without progression of the underlying retinal detachment; or (b) Decrease in visual acuity from baseline to no light perception.
4. Any serious adverse event (SAE) that occurs within the first 14 days following ONL1204 injection that, in the opinion of the investigator, is related to ONL1204.

In total 7 out of 8 patients (4 patients receiving 25 (micrograms (ug) and 4 patients receiving 50 ug of ONL1204) experienced a total of 20 adverse events. Most were mild, and study procedure or surgery related. After evaluating the data for each cohort, the SRC unanimously recommended proceeding with dose escalation to a 100 ug dose.

### Outcomes

All patients receiving doses comprising 25 ug and 50 ug of ONL1204 showed an improvement in visual outcomes (e.g., visual acuity). Table 1 shows study data for Snellen best corrected visual acuity (BCVA). Table 2 shows study data for logMAR best corrected visual acuity (BCVA). As demonstrated by Table 1 and Table 2, an improvement in visual acuity was observed for all patients receiving 25 ug and 50 ug of ONL1204, and across all patient ages within the study. Table 3 shows the time between date of (i) the onset of symptoms or (ii) central vision loss and the date of screening. Table 3 shows the time between the date of (i) the onset of symptoms or (ii) central vision loss and the date of administering ONL1204. As demonstrated by Table 3, improved visual outcomes were observed for all patients receiving ONL1204, even when ONL1204 was administered to patients having greater than 10 days of central vision loss. Table 4 shows the time from administering ONL1204 to performing surgery. Table 4 also shows between the date of (i) the onset of symptoms or (ii) central vision loss and the date of surgery. As demonstrated by Table 4, improved visual outcomes were observed for all patients even when the time from administering ONL1204 to surgery was 3 days or greater (ranging up to 7 days). As demonstrated by Table 4, improved visual outcomes were observed for all patients receiving ONL1204, even when ONL1204 was administered to patients having greater than 15 days of central vision loss prior to the time of surgery to reattach the retina. Table 5 shows the extent (1-12, 12 being full) and height of detachment. As demonstrated by Table 5, improved visual outcomes were observed for all patients receiving ONL1204, across a range of detachment heights and extent of detachments.

Table 6 shows summary statistics for all patients, including sample size (n), mean, and standard deviation (sd). By way of a comparison to surgery alone, a prospective population-based study of 291 Scotland patients with macula-off rhegmatogenous retinal detachments (RRD) without pre-existing retinal disease who had successful repair after one operation reported a baseline mean logMAR visual acuity of 1.71 with follow up mean logMAR visual acuities of 0.87, 0.66, 0.65, and 0.57 at 6 weeks, 3 months, 6 months, and 12 months post-surgery, respectively (Mitry et al. “Long-term visual acuity and the duration of macular detachment: findings from a prospective population-based study” Br J Ophthalmol. 2013 February; 97(2):149-52. doi: 10.1136/bjophthalmol-2012-302330. Epub 2012 Nov. 17. PMID: 23159447). The study further reported for patients having a detachment less than or equal to 8 days a baseline mean logMAR visual acuity of 1.73 with follow up mean logMAR visual acuities of 0.81, 0.60, 0.55, and 0.45 at 6 weeks, 3 months, 6 months, and 12 months post-surgery, respectively. For patients having a detachment greater than 8 days, the study reported a baseline mean logMAR visual acuity of 1.67 with follow up mean logMAR visual acuities of 0.98, 0.82, 0.86, and 0.79 at 6 weeks, 3 months, 6 months, and 12 months post-surgery, respectively.

Table 7 shows the change in cytokine levels from time of ONL1204 administration to surgery. As demonstrated by Table 7, administration of ONL1204 resulted in a mean decrease in MCP1, ICAM, IL1RA, and IL18.

TABLE 1

| | | Dose | BCVA Snellen study eye | | | |
|---|---|---|---|---|---|---|
| Patient ID | Age | (ug) | baseline | week 6 | week 9 | week 24 |
| 001^{#} | 68 | 25 | 20/160 | 20/40 | 20/25 | 20/60 |
| 002^{#} | 62 | 25 | 20/100 | 20/32 | 20/25 | 20/32 |
| 005 | 70 | 25 | 20/200 | 20/125 | 20/100 | 20/100 |
| 002-2* | 70 | 25 | HM | 20/125 | 20/125 | 20/100 |
| 006* | 52 | 50 | CF | 20/50 | 20/50 | 20/50 |
| 007^{%} | 55 | 50 | HM | 20/62.5 | 20/80 | 20/125 |
| 008 | 68 | 50 | CF | 20/50 | — | 20/32 |
| 009^{#%} | 43 | 50 | CF | 20/80 | 20/80 | 20/80 |
| 010*^{%} | 49 | 100 | HM | 20/125 | 20/200 | 20/63 |
| 003 | 56 | 100 | 20/100 | 20/100 | 20/80 | 20/100 |
| 001-2*^{#} | 43 | 100 | 20/200 | 20/200 | HM | 20/400 |
| 002-3*^{%} | 84 | 100 | HM | 20/800 | 20/400 | 20/80 |
| 004-2 | 65 | 200 | 20/160 | 20/50 | 20/40 | 20/32 |
| 012 | 70 | 200 | HM | 20/100 | 20/80 | — |

(HM) = hand motion
(CF) = counting fingers
*= received silicone oil replacement,
^{#}= patient demonstrating cataract or potential cataract
^{%}= re-detachment observed in patient
(—) = data not available

TABLE 2

BCVA logMAR study eye

| Patient ID | baseline | week 6 | week 9 | week 24 |
|---|---|---|---|---|
| 001 | 0.68 | 0.34 | 0.14 | 0.5 |
| 002 | 0.6 | 0.18 | 0.12 | 0.2 |
| 005 | 1.08 | 0.82 | 0.64 | 0.68 |
| 002-2 | 3 | 0.82 | 0.78 | 0.7 |
| 006 | 2 | 0.38 | 0.34 | 0.34 |
| 007 | 3 | 0.46 | 0.6 | 0.76 |
| 008 | 2 | 0.38 | — | 0.2 |
| 009 | 2 | 0.52 | 0.56 | 0.64 |
| 010 | 3 | 0.8 | 1 | 0.5 |
| 003 | 0.7 | 0.7 | 0.6 | 0.7 |
| 001-2 | 1 | 1 | 3 | 1.3 |
| 002-3 | 3 | 1.6 | 1.4 | 0.6 |
| 004-2 | 0.92 | 0.4 | 0.36 | 0.2 |
| 012 | 3 | 0.7 | 0.6 | — |

— = data not available

TABLE 3

| | Time from reported symptom | Time from loss of central |
|---|---|---|
| | onset to administering | vision to administering |
| Patient ID | ONL1204 (days) | ONL1204 (days) |
| 001 | 19 | 15 |
| 002 | 42 | 14 |
| 005 | 22 | 15 |
| 002-2 | 27 | 20 |
| 006 | 14 | 13 |
| 007 | 12 | 12 |
| 008 | 9 | 9 |
| 009 | 28 | 10 |
| 010 | 106 | 12 |
| 003 | 8 | 8 |
| 001-2 | 29 | 10 |
| 002-3 | 9 | 7 |
| 004-2 | 32 | 19 |
| 012 | 12 | 10 |

TABLE 4

| | Time from | Time from | |
|---|---|---|---|
| | administering | reported symptom | Time from loss |
| Patient | ONL1204 to | onset of symptoms | of central vision |
| ID | surgery (days) | to surgery (days) | to surgery (days) |
| 001 | 3 | 22 | 18 |
| 002 | 4 | 46 | 18 |
| 005 | 6 | 28 | 21 |
| 002-2 | 4 | 31 | 24 |
| 006 | 3 | 17 | 16 |
| 007 | 7 | 19 | 19 |
| 008 | 3 | 12 | 12 |
| 009 | 4 | 32 | 14 |
| 010 | 3 | 109 | 15 |
| 003 | 3 | 11 | 11 |
| 001-2 | 6 | 35 | 16 |
| 002-3 | 5 | 14 | 12 |
| 004-2 | 3 | 35 | 22 |
| 012 | 8 | 20 | 18 |

TABLE 5

| Patient | Extent of Detachment | Height of Detachment |
|---|---|---|
| ID | (1-12, clock hours) | at central macula (mm) |
| 001 | 6 | 1 |
| 002 | 11 | 1 |
| 005 | 12 | 1 |
| 002-2 | 7 | 11 |
| 006 | 7 | 5 |
| 007 | 4 | 2 |
| 008 | 5 | 3 |
| 009 | 6 | 4 |
| 010 | 12 | 2 |
| 003 | 3 | 1 |
| 001-2 | 7 | — |
| 002-3 | 6 | — |
| 004-2 | 7 | 1 |
| 012 | 7 | 2 |

— = data not available

TABLE 6

| | n | mean | sd |
|---|---|---|---|
| Baseline | 14 | 1.86 | 0.99 |
| Week 6 | 14 | 0.65 | 0.36 |
| Week 9 | 13 | 0.78 | 0.74 |
| Week 24 | 13 | 0.56 | 0.30 |

TABLE 7

| | Percent Change (%) from time of administering ONL1204 to surgery | | | |
|---|---|---|---|---|
| Patient ID | MCP1 | ICAM1 | IL1RA | IL18 |
| 001 | −41.4 | −82.4 | −51.0 | −60.5 |
| 002 | −6.02 | −42.7 | −57.4 | −22.5 |
| 002-2 | −35.9 | −86.2 | −99.3 | −50.8 |
| 006 | 11.6 | 0 | −88.3 | 0 |
| 007 | −3.12 | 64.0 | −60.0 | 0 |
| 008 | −48.4 | −100 | −94.4 | −100 |
| 010 | −9.5 | −43.9 | 8.5 | −33.7 |
| 003 | 78.5 | 24.9 | −46.1 | 0 |
| 001-2 | 500 | 22.7 | −65.2 | 66.0 |
| 004-2 | −2.4 | 0 | −26.3 | 0 |

data not available or adverse event not related to drug was observed for Patient ID 005, Patient ID 009, Patient ID 002-3, and Patient ID 012

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

SEQUENCES

| SEQ ID NO | SEQUENCE |
|---|---|
| 1 | HHIYLGAVNYIY |
| 2 | YIYNVAGLYIHH |
| 3 | HHIYLGAVNYIY |
| 4 | YIYNVAGLYIHH |
| 5 | YIYNVAGLYIHH |
| 6 | YVYNVAGLYVHH |
| 7 | YVYNVAGLYVHH |
| 8 | YIYNVAGLYIHH |
| 9 | YVYNVAGLYVHH |

1. A method of improving visual function (e.g., BCVA) in an eye of an individual having an ocular disease or disorder, comprising:
administering a peptide (e.g., a composition comprising the peptide) comprising an amino acid sequence HHIYLGAVNYIY or a variant sequence thereof, or a pharmaceutically-acceptable salt of the peptide, to the eye of the individual.

2. The method of claim 1, wherein the ocular disease or disorder comprises a loss in visual function.

3. The method of any one of claims 1-2, wherein the ocular disease or disorder comprises photoreceptor cell death or a symptom thereof (e.g., a loss in visual function).

4. The method of any one of claims 1-2, wherein the ocular disease or disorder comprises retinal pigment epithelial cell death or a symptom thereof (e.g., a loss in visual function).

5. The method of any one of claims 1-4, wherein the ocular disease or disorder comprises degeneration of the macula.

6. The method of any one of claims 1-5, wherein the visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and light/dark adaptation.

7. The method of any one of claims 1-6, wherein the method comprises improving visual function without performing surgery.

8. The method of any one of claims 1-6, wherein the method further comprises performing surgery (e.g., standard of care surgery) on the eye of the individual.

9. The method of claim 8, wherein the surgery is performed after administering the peptide or the variant sequence thereof, or the pharmaceutically-acceptable salt of the peptide.

10. The method of claim 8, wherein the surgery comprises retinal reattachment surgery.

11. The method of any one of claims 8-10, wherein the surgery comprises a vitrectomy.

12. The method of any one of claims 1-11, wherein improving visual function comprises improving visual function as compared to a baseline visual function prior to the administering peptide or the variant sequence thereof, or the pharmaceutically-acceptable salt of the peptide.

13. A method of treating macular degeneration having geographic atrophy in an eye of an individual, comprising:
administering a peptide (e.g., a composition comprising the peptide) comprising an amino acid sequence HHIYLGAVNYIY or a variant sequence thereof, or a pharmaceutically-acceptable salt of the peptide, to the eye of the individual.

14. A method of treating a loss in visual function (e.g., BCVA) in an eye of an individual having macular degeneration, comprising:
administering a peptide (e.g., a composition comprising the peptide) comprising an amino acid sequence HHIYLGAVNYIY or a variant sequence thereof, or a pharmaceutically-acceptable salt of the peptide, to the eye of the individual.

15. The method of any one of claims 13-14, wherein treating comprises improving visual function.

16. The method of claim 15, wherein improving visual function is compared to a baseline visual function prior to administering the peptide or the variant sequence thereof, or the pharmaceutically-acceptable salt of the peptide.

17. The method of any one of claims 13-16, wherein the macular degeneration comprises age-related macular degeneration.

18. The method of any one of claims 13-17, wherein the macular degeneration comprises geographic atrophy.

19. The method of any one of claims 13-18, wherein visual function comprises one or more measurements selected from the group consisting of: best corrected visual acuity (e.g., letters read), field of vision, contrast sensitivity, binocular function, low luminance acuity, low contrast acuity, color vision, perimetry, threshold sensitivity, reading speed, and/or light/dark adaptation.

20. A method of treating retinal detachment in an eye of an individual, comprising:
administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual.

21. A method of treating a loss in visual function in an eye of an individual having a retinal detachment, comprising:
administering a peptide (e.g., a composition comprising the peptide) comprising an amino acid sequence HHIYLGAVNYIY or a variant sequence thereof, or a pharmaceutically-acceptable salt of the peptide, to an eye of an individual, and performing surgery (e.g., standard of care surgery) on the eye of the individual.


SEQUENCE LISTING (18 sequences)

SEQ ID NO: 1 - protein, 12 aa, synthetic construct
HHIYLGAVNYIY

SEQ ID NO: 2 - protein, 12 aa, synthetic construct
YIYNVAGLYIHH

SEQ ID NO: 3 - protein, 12 aa, synthetic construct
HHIYLGAVNYIY

SEQ ID NO: 4 - protein, 12 aa, synthetic construct
YIYNVAGLYIHH

SEQ ID NO: 5 - protein, 12 aa, synthetic construct
YIYNVAGLYIHH

SEQ ID NO: 6 - protein, 12 aa, synthetic construct
YVYNVAGLYVHH

SEQ ID NO: 7 - protein, 12 aa, synthetic construct
YVYNVAGLYVHH

SEQ ID NO: 8 - protein, 12 aa, synthetic construct
YIYNVAGLYIHH

SEQ ID NO: 9 - protein, 12 aa, synthetic construct
YVYNVAGLYVHH

SEQ ID NO: 10 - protein, 14 aa, synthetic construct
  VARIANT 13..14: note = This region may encompass Ala-His-amide, Gly-His-amide, [D]Ala-[D]-His-amide or Gly-[D]-His-amide
HHIYLGAVNYIYXX

SEQ ID NO: 11 - protein, 14 aa, synthetic construct
  VARIANT 13..14: note = This region may encompass Ala-His-amide, Gly-His-amide, [D]Ala-[D]-His-amide or Gly-[D]-His-amide
YIYNVAGLYIHHXX

SEQ ID NO: 12 - protein, 12 aa, synthetic construct
  SITE 12: note = Tyrosine amide
HHIYLGAVNYIY

SEQ ID NO: 13 - protein, 12 aa, synthetic construct
  SITE 1: note = D-Tyrosine
  SITE 2: note = D-Isoleucine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-Isoleucine
  SITE 11: note = D-Histidine
  SITE 12: note = D-Histidine amide
YIYNVAGLYIHH

SEQ ID NO: 14 - protein, 12 aa, synthetic construct
  SITE 1: note = D-Tyrosine
  SITE 2: note = D-alloisoleucine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-alloisoleucine
  SITE 11: note = D-Histidine
  SITE 12: note = D-Histidine amide
YXYNVAGLYXHH

SEQ ID NO: 15 - protein, 12 aa, synthetic construct
  SITE 1: note = D-Tyrosine
  SITE 2: note = D-Valine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-Valine
  SITE 11: note = D-Histidine
  SITE 12: note = D-Histidine amide
YVYNVAGLYVHH

SEQ ID NO: 16 - protein, 12 aa, synthetic construct
  SITE 1: note = D-DesaminoTyrosine
  SITE 2: note = D-Valine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-Valine
  SITE 11: note = D-Histidine
  SITE 12: note = D-Histidine amide
XVYNVAGLYVHH

SEQ ID NO: 17 - protein, 12 aa, synthetic construct
  SITE 1: note = D-HydroxydesaminoTyrosine
  SITE 2: note = D-alloisoleucine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-alloisoleucine
  SITE 11: note = D-Histidine
  SITE 12: note = D-Histidine amide
XXYNVAGLYXHH

SEQ ID NO: 18 - protein, 12 aa, synthetic construct
  SITE 1: note = D-DesaminoTyrosine
  SITE 2: note = D-Valine
  SITE 3: note = D-Tyrosine
  SITE 4: note = D-Asparagine
  SITE 5: note = D-Valine
  SITE 6: note = D-Alanine
  SITE 7: note = D-Glycine
  SITE 8: note = D-Leucine
  SITE 9: note = D-Tyrosine
  SITE 10: note = D-Valine
  SITE 11: note = D-Histidine
  SITE 12: note = D-Histidine piperazine amide
XVYNVAGLYVHH